(19) United States
(12) Patent Application Publication
(10) Pub. No.: US 2010/0297009 A1
(43) Pub. Date: Nov. 25, 2010

(54) SELF-ASSEMBLED POLYHEDRAL MULTIMERIC CHEMICAL STRUCTURES

(75) Inventors: Arthur Olson, Del Mar, CA (US); Ehud Keinan, Timrat (IL)

Correspondence Address: MARTIN D. MOYNIHAN d/b/a PRTSI, INC., P.O. BOX 16446, ARLINGTON, VA, 22215, US

(73) Assignee: TECHNION RESEARCH & DEVELOPMENT FOUNDATION LTD., Technion City, Haifa (IL)

(21) Appl. No.: 12/450,114

(22) PCT Filed: Mar. 13, 2008
(86) PCT No.: PCT/IL2008/000353

§ 371 Date: Aug. 9, 2010

Related U.S. Application Data
(60) Provisional application No. 60/906,899, filed on Mar. 13, 2007.

(51) Int. Cl. A61K 49/00; A61K 9/16; A61K 39/00; A61K 47/00; C07D 403/14; C07D 471/22; C07D 487/22

(52) U.S. Cl. 424/9.1; 424/490; 424/184.1; 514/788; 544/296; 546/27; 548/417; 977/773; 977/906; 977/920; 977/736; 977/737

(57) ABSTRACT

Self-assembled, closed and hollow chemical multimer structures having a dodecahedral morphology, composed of chemical monomers having a structurally symmetric core which possess a 5-fold rotational symmetry, are provided. Also provided are methods of creating such chemical monomers, methods of creating such chemical multimer structures and compositions comprising these chemical multimer structures. Also provided are uses of these chemical multimer structures in applications such as drug delivery, imaging, immunization, formation of plastic crystals and nanoparticle matrices and other medical and material science applications.

### FIELD AND BACKGROUND OF THE INVENTION

The present invention relates generally to design and generation of self-assembled multi-molecular chemical structures, and, more particularly, to closed and hollow chemical icosahedral structures which can self-assemble and disassemble in a stochastic process governed, for example, by pre-determined chemical and/or physical environmental conditions.

Molecular self assembly of closed and hollow structures and its most intuitive use for molecular encapsulation and the formation of uniform molecular spheroid nanoparticles have long been an intellectual and practical challenge, and the focus of many experimental studies.

A higher goal is set when the closed and hollow, and possibly encapsulating structure, is required not only to self-assemble under specific conditions but also disassemble under different specific conditions, hence constitute a reversibly space encapsulating structure. An even higher goal is set when the closed and hollow-assembled structures are uniform and capable to pack themselves into well ordered 2- or 3-dimensional lattices, owing to the inherent symmetry.

Reversible encapsulation by closed and hollow self-assembled structures isolates discrete volumes of space wherein molecules can be shielded temporarily from the environment, either in solid, gaseous or solubilized phase. The self-assembly process may depend on the presence of the molecule(s) to be encapsulated, yet may not, allowing the option to enclose an “empty space”, being, for example, the environmental media, such as solvent or gas, or vacuum.

Many attempts have been made at achieving reversible molecular encapsulation. Some of these attempts were successful, such as reported, for example, by Rebek [Rebek, J., Angew. chem. int. ed., 2005, 44, 2068-2078]. In this report, Rebek describes self-assembled complexes made of one or more molecules and a capsule encapsulating the molecule(s). These complexes form when, and only when, the spaces inside the capsules are appropriately filled with the molecule(s), wherein weak intermolecular forces hold these self-assemblies together and allow equilibration of the encapsulation complexes at ambient temperatures and pressures in liquid phase. However, these complexes lack the generality to encapsulate various objects and empty space, and lack the capacity to form 2- or 3-dimensional lattices.

The sphere is the simplest finite closed and hollow structure that partitions space and can be packed tightly in 2- and in 3-dimensional lattices. Nature uses spheres at all scales in both the inanimate and living world for the basic physical property of encapsulation. One of nature's minute marvels of molecular architecture and one of the most efficient self-assembled spherical encapsulation devices is the complex arrangements of macromolecules in a spherical virus, and more specifically in the spherical viral capsid.

It is not a coincidence that many viral capsids are spheroids, or have at least one circular dimension (cylindrical, helical, etc.). In 1956, Crick and Watson proposed a theoretical model suggesting that there are certain common features and general principles of architecture that apply to all spherical virus capsids, which later have been amply confirmed and universally accepted. Crick and Watson pointed out that the nucleic acid in small virions was probably insufficient to code for more than a few sorts of protein molecules of limited size, and that the only reasonable way to build a proteinous shell was to use a plurality of the same type of molecule, thereby constituted their theory of identical subunits. Further, they proposed that the subunits would be packed so as to provide each with an identical environment, hence the subunits would pack symmetrically. This theoretical model was shortly after confirmed and it became evident that the occurrence of icosahedral features in quite unrelated viruses was not a matter of chance selection but that icosahedral symmetry is preferred in virus structure, and that evolutionary processes selected spherical viral capsids so as to enclose space by utilizing the geometry of the icosahedron and thus exploit the economy of this form in terms of both surface-to-volume ratio and genetic efficiency of subunit-based symmetric assembly.

Thus, the formation of a viral capsid is a classical example of a naturally occurring and stochastically controlled process of an assembly of a multi-component system, wherein multiple individual units, termed capsomers, self-assemble to form the capsid.

A capsomer is often a complex yet symmetric multimeric structure which is comprised of several sub-structural units, each made of several protein chains having distinct sequences (viral coat proteins). A capsomer is a protein-based subunit of a viral capsid, designed to have strong affinity to other identical capsomers so as to form a particular structure and, upon reaching a minimal number of subunits, self-assemble to form that structure, namely the capsid.

One of the most known spherical closed and hollow structures having an icosahedral symmetry is the spheroidal fullerene or buckminsterfullerene, better known as the buckyball, for which Kroto, Curl, and Smalley were awarded the 1996 Nobel Prize in Chemistry for their roles in the discovery of this class of compounds. However, buckyballs are rigid balls made substantially of carbon atoms held together by covalent bonds, and hence form practically irreversible encapsulating structures. Furthermore, fullerenes are typically not formed from a set of multi-atom subunits but rather from bulk carbon, and the fully formed fullerene is almost unreactive, hence fullerene are not suitable for derivatization and alterations, particularly once they are fully formed.

Nano-scale self-assembled hollow molecular structures of substantially inorganic composition have been studies and reported. Such structures, typically made of metal oxides and other substances, can be viewed as mimicking a viral capsid by having highly symmetric spherical forms, and although most of these structures self-assemble irreversibly, some have been design to assemble and disassemble under various conditions. Such systems are reported by, for example, Muller et al. [Muller et al., Angew. chem. int. ed., 2003, 42, 2085-2090 and Angew. chem. int. ed., 2004, 43, 4466-4470], who teaches of nano-porous capsules that can uptake and release metal ions such as lithium ions. Although reported to take various sizes, these capsules lack the capacity to encapsulate organic compounds even of relatively small size due to instability or solvation incompatibility. Furthermore, the inorganic composition of these capsules limits their use due to toxicity and other bio- and physiological considerations.

U.S. Pat. No. 6,965,026 discloses nano-scale polyhedron-shaped molecules that have molecular building blocks connected at their vertices, which are produced by a self-assembly reaction. Although the resulting molecules are said to be porous, chemically robust and contain chemically accessible sites on their facets, these structures fail to possess the capacity to disassemble, and the use thereof is generally limited to non-physiological conditions. Furthermore, these structures could not be used to encapsulate sensitive substances such as drugs and other biologically and pharmaceutically active agents since they are prepared under conditions that will damage most organic substances.

U.S. Pat. No. 6,531,107 discloses closed and hollow spherical compounds, however, much like fullerene (C₆₀ buckyball), these compounds do not exhibit reversible assembly and disassembly capacity once formed, and are mostly formed under chemical conditions that are non-viable for encapsulation of organic substances such as pharmaceutically active agents and drugs.

There is thus a widely recognized need for, and it would be highly advantageous to have a uniform, closed and hollow chemical structure, which can self-assemble under various conditions, devoid of the above limitations.

Closed and hollow reversibly self-assembled structures that can encapsulate organic substances such as biologically and pharmaceutically active agents, can be considered as ideal drug delivery systems, which offer solutions to the limitations commonly associated with such systems, as follows.

Most of the currently known drug delivery systems are based on carriers made of macromolecules such as polysaccharides and proteins, synthetic polymers and particles of inorganic origins. These systems are based on covalently binding drug molecules to the carrier, or encapsulating small amounts of a composition containing the drug within the carrier. The carrier can further be either intrinsically tissue-specific or can be rendered such by, for example, attaching to the carrier a moiety that has an affinity to the targeted tissue.

The primary role of a carrier for drug delivery is to provide an isolating matrix that protects the drug at initial stages of the drug administration process. Such an isolating protection is required so as to avoid a premature contact of the drug with e.g., body fluids, and thereby circumvent premature decomposition of the drug. The carrier is further designed so as to release the drug at a desired site (e.g., a desired organ or tissue).

Most of the drug delivery systems employ the concept of slow release of the drug under physiological conditions, wherein the carrier is slowly degraded, resorbed, dissolved or otherwise erodes away, exposing the drug to the environment. When the system includes a targeting moiety, the process of drug exposure is desirably designed to occur near or at the drug target.

Most of the presently known drug delivery systems are limited by one or more of the above factors. Thus, for example, in many systems the quantity and range of drugs that can be incorporated into the carrier matrix is limited and often does not conform to the required amount and/or range of the loaded drug. Many systems are characterized by poor targeting capacity and hence result in poor efficacy as well as undesired toxicity caused by interaction of the drug with other cells or tissues. In addition, many systems are limited by toxicity of the carrier, which results, for example, from toxic components composing the carrier or from toxic degradation by-products. Furthermore, technical and practical problems are often involved in preparation of the drug-carrier matrix, leading to a laborious preparation process, whereby often, the preparation process involves inactivation of the drug.

Liposomes are microscopic vesicles formed from natural lipid constituents, such as phospholipids, which are devoid of the limitations associated with protein microspheres, and hence have been widely used as a drug delivery matrix. Being hollow like micelles, pharmacologically active agents can be entrapped in the liposomes for subsequent delivery. A comprehensive review of liposome technology is found, for example, in Gregoriadis, G., Liposome Technology (CRC Press, Roca Baton, Fla., 1984); and Gregoriadis, G., Trends in Biotechnology (1985) 3:235.

Although the use of liposomes as a drug delivery system alleviates some of the limitations described above, this drug delivery technique is still rather limited. Thus, liposomes often lack an effective targeting capacity and further, often exhibit poor stability during storage. Large scale production and manufacturing of liposome-based drug delivery systems have also been found to be problematic.

In view of the limitations associated with common drug delivery systems, it has been recognized that an effective drug delivery system may combine benign yet durable and synthetically versatile chemical components which readily assemble and disassemble in various chemical environments, whereby by these assembling/disassembling, encapsulation and release of a drug could be effected. A set of such components may assemble to construct a spheroid shape around one or more drug molecules.

In an attempt to overcome the above-described limitations associated with many commonly used drug delivery systems, researchers have employed the advantages exhibited by natural encapsulating systems such as the viral capsids, and shown substance delivery using native and modified viral particles. Thus, WO 1991/06658, for example, describes the use of retroviral vectors carrying RNA sequences that encode tumor necrosis factor, interleukin-2 and multiple drug resistance proteins.

U.S. Pat. Nos. 5,071,651 and 5,503,833 teach compositions and methods for preparing and delivering encapsulated biologically active agents to specific cell types by encapsulating these agents in the VP6 inner capsid protein of rotavirus which are then delivered to selected cells, tissues and organs. According to the teachings of these patents, targeting agents, such as protein, peptide and carbohydrate antigens, can be linked to the surface of the VP6 sphere.

U.S. Pat. Nos. 6,046,173, 6,261,765, 6,416,945, 6,613,749 and 6,962,777 also teach uses of native and modified viral components mainly as vehicles for improved purification, delivery of active agents and exogenous material transfer.

However, viral components, being substantially proteinaceous, suffer from many drawbacks which are common to many protein-based pharmaceuticals, such as mechanical and chemical instability, toxicity profile and technical preparation difficulties.

Hence, a need still exists for a drug delivery system which would employ the advantages exhibited by the viral self-assembled spheroid encapsulation system, yet would exhibit a favorable preparation conditions with respect to both the drug chemical and physical sensitivity and the stability of the integrated delivery system once formed, would present a favorable pharmacokinetic profile, and would exhibit target-specificity.

Such a system should mimic a viral capsid while preserving the hollow, spherical, self-assembled encapsulating structure, while utilizing stable and viable chemical component.

Uniform and reversibly self-assembled nanoscale structures which can encapsulate other substances therein or be derivatized so as to have other substances exteriorly attached thereon, can be considered as ideal nanoparticles, which can be used in a myriad of applications where nanoparticles are used.

Nanoparticles are larger than molecules but smaller than bulk solids and therefore frequently exhibit unique physical and chemical properties due to their size and uniform morphology (shape). A bulk material is typically characterized by constant physical properties which are independent from its size, while nanoscale particles, sometimes of the same substance, exhibit different characteristics. Nanoparticles often exhibit properties which are not observed in bulk such as quantum confinement of semiconductor nanoparticles, surface plasmon resonance (SPR) in particular metallic nanoparticles and superparamagnetism in magnetic nanoparticles.

Given that a nanoparticle is an intermediate state between single molecules and a solid bulk and is practically all surface and substantially no interior, the physical, chemical and mechanical properties of a nanocrystal can be finely controlled as it grows in size and varies in morphology. For example, by finely controlling the size and surface of a nanoparticle, properties such as the band-gap, conductivity, crystal lattice and symmetry and melting temperature, can be tuned.

In recent years there has been a major progress in methods for controlling the growth of nanoparticles of semiconducting, metallic, magnetic and oxide materials. Shape-control growth methods have enabled the preparation of nanoparticles in various forms such as dots, rods, tetrapods and more. It has been recognized that the size-, composition- and shape-dependent properties of such nanoparticles can be harnessed for a variety of applications in areas ranging from biological fluorescent tagging, medical devices and therapeutics to light emitting diodes, lasers and chemical catalysts.

Nanoparticles are difficult to produce uniformly in terms of shape, size and composition, and while the photo-electronic characteristics of nanoparticles depend on their shape and surface, any undesired and ill-controlled chemical or physical change may adversely affect their characteristics. Hence, there is currently a technological limit to produce nanoparticles which are uniform in terms of size, shape and composition, and further are of the order of magnitude of 2 nm to 10 nm.

Closed and hollow self-assembled chemical structures, designed so as to mimic, for example, viral capsids, can be advantageously utilized in applications such as drug delivery and nanoparticles systems, as well as in any application in which the beneficial characteristics of such systems can be exploited, while overcoming the limitations associated with currently known methodologies.

### SUMMARY OF THE INVENTION

According to one aspect of the present invention there is provided a method of creating a closed, hollow and self-assembled chemical multimer structure having a dodecahedral morphology, the method comprising: (a) providing a plurality of chemical monomers that form the self-assembled chemical multimer structure, wherein each of the chemical monomers comprises a structurally symmetric core having a 5-fold rotational symmetry, and a plurality of at least one type of associating groups, the plurality of associating groups being symmetrically positioned at a periphery of the structurally symmetric core, whereas the chemical monomers have structural complementarity to one another so as to form the closed, hollow and self-assembled chemical multimer structure upon occurrence of an associative proximity and orientation of the associating groups; and (b) subjecting the plurality of the chemical monomers to conditions allowing the chemical monomers to associate therebetween via the associating groups, thereby creating the closed, hollow and self-assembled chemical multimer structure.

According some embodiments of the invention described below, the chemical monomers in the plurality of chemical monomers are identical to one another.

According to some embodiments the core of each of the chemical monomers in the plurality of chemical monomers is the same.

According to some embodiments the at least one type of the associating groups in each of the chemical monomers is the same.

According to some embodiments a position at which each of the associating groups is attached to each of the chemical monomers is the same in each of the chemical monomers.

According to some embodiments the plurality of chemical monomers comprises at least two different chemical monomers.

According to some embodiments the at least two different chemical monomers differ from one another by at least one type of the associating groups, by a position of at least one of the associating groups and/or by a number of the associating groups in each of the different chemical monomers.

According to some embodiments the plurality of chemical monomers comprises from two to four types of chemical monomers that are different from one another.

According to some embodiments the chemical monomers are associated therebetween via at least one interaction selected from the group consisting of a hydrogen bond interaction, an ionic interaction, a metal-coordination interaction and a hydrophobic interaction.

According to some embodiments the associating groups are selected capable of forming a directional (polar) bond.

According to some embodiments the bond is selected from the group consisting of a hydrogen bond, a salt-bridge bond, a metal-ligand coordination bond, a hydrophobic interaction bond and a combination thereof.

According to some embodiments the associating groups are selected capable of forming a chemically-reversible bond.

According to some embodiments the associating groups are selected such that at least one type of the associating groups in one chemical monomer of the plurality of chemical monomers and at least one type of the associating groups in another chemical monomer in the plurality of chemical monomers are capable to interact therebetween via the at least one interaction.

According to some embodiments the associating groups are selected capable of forming a biocleavable bond.

According to some embodiments the associating groups are selected capable of forming a biostable bond.

According to some embodiments the structurally symmetric core comprises at least one aromatic moiety.

According to some embodiments the structurally symmetric core comprises a corannulene moiety.

According to some embodiments the at least one type of associating groups in each of the chemical monomers is independently selected from the group consisting of amine, hydroxyl, thiol, alkoxy, aryloxy, thioalkoxy, thioaryloxy, aryl, heteroaryl, heteroalicyclic, halide, carbonyl, carboxylate, amide, phosphate, phosphonate, phosphine, sulfate, sulfonate, sulfite, lactam, guanidine, purine, pyrimidine, nitrile, carbamyl, thiocarbamyl, sulfonamide, azide, hydroxamate, and a metallic element.

According to another aspect of the present invention there is provided a closed, hollow and self-assembled chemical multimer structure having a dodecahedral morphology comprising a plurality of chemical monomers, as described herein, wherein each of the chemical monomers comprises a structurally symmetric core having a 5-fold rotational symmetry, and a plurality of at least one type of associating groups, the plurality of associating groups being symmetrically positioned at a periphery of the structurally symmetric core, whereas the chemical monomers have structural complementarity to one another, thus forming the closed, hollow and self-assembled chemical multimer structure via associative proximity and orientation of the associating groups.

According to still another aspect of the present invention there is provided a composition comprising the closed, hollow and self-assembled chemical multimer structure described herein and at least one active agent being attached to and/or encapsulated in the chemical multimer structure.

According to further features in embodiments of the invention described below, the composition comprising a plurality of the at least active agents, each of the active agents being independently attached to and/or encapsulated in the chemical multimer structure.

According to some embodiments the at least one active agent is selected from the group consisting of a pharmaceutically active agent, a labeling agent, a surface-modifying agent, a chemical compound, a metal and a nanoparticle.

According to some embodiments the pharmaceutically active agent is selected from the group consisting of a therapeutically active agent and a targeting moiety.

According to some embodiments the therapeutically active agent is selected from the group consisting of a drug, a chemotherapeutic agent, an amino acid, a peptide, a polypeptide, a protein, an antigen, an antibody, a nucleic acid, a nucleic acid construct, a gene, a cardiovascular agent, a co-factor, a cytokine, a growth factor, a heparin, a hormone, a ligand, a lipid, a metabolite, a phospholipid, a prostaglandin, a receptor agonist, a receptor antagonist, a toxin, a vitamin, an agonist, an analgesic, an antagonist, an antibiotic, an antidepressant, an anti-diabetic agent, an anti-histamine, an anti-hypertensive agent, an anti-inflammatory drug, an anti-metabolic agent, an antimicrobial agent, an antioxidant, an anti-platelet agent, an anti-proliferative agent, an anti-psychotic agent, an antisense, an anti-thrombogenic agent, an enzyme, an epitope, an immunoglobulin, an inhibitor, an oligonucleotide and any combination thereof.

According to some embodiments the composition comprising at least one therapeutically active agent encapsulated in or attached to the multimer structure and at least one targeting moiety attached to the multimer structure.

According to some embodiments the associating groups in the chemical monomers forming the multimer structure are selected capable of forming a biocleavable bond.

According to some embodiments a composition as described hereinabove is identified for use in drug delivery.

According to further features in embodiments of the invention described below, the at least one therapeutically active agent is selected from the group consisting of an oligonucleotide, a nucleic acid, a nucleic acid construct and an antisense and further wherein the at least one targeting moiety comprises a cell-internalizing moiety.

According to some embodiments the associating groups in the chemical monomers forming the multimer structure are selected capable of forming a biocleavable bond, the biocleavable bond being cleavable by a cellular component.

According to some embodiments, a composition as described hereinabove is identified for use in gene therapy.

According to further features in embodiments of the invention described below, the composition comprising at least one therapeutically active agent attached to the multimer structure, the at least one therapeutically active agent being an epitope.

According to some embodiments the composition comprising a plurality of the epitopes.

According to some embodiments, a composition as described hereinabove is identified for use in immunization.

According to some embodiments of the invention described below, the active agent is a labeling agent, and the composition is identified for use in diagnosis.

According to some embodiments the labeling agent is selected from the group consisting of a fluorescent agent, a radioactive agent, a magnetic agent, a chromophore, a phosphorescent agent and a heavy metal cluster.

According to some embodiments such a composition further comprising a pharmaceutically active agent encapsulated therein or attached thereto.

According to some embodiments of the invention described below, the surface-modifying agent is selected from the group consisting of a hydrophobic moiety, a charged moiety, a hydrophilic moiety, and an amphiphilic moiety.

According to some embodiments of the invention described below, the active agent is a chemical compound, the chemical compound being encapsulated in the multimer structure.

According to some embodiments such a composition is identified for use in an analytical method for determining a chemical feature of the chemical compound.

According to some embodiments such a composition the analytical method is mass spectroscopy and the chemical compound is non-ionized.

According to further features in embodiments of the invention described below, the active agent is a nanoparticle, the nanoparticle being encapsulated in the multimer structure.

According to some embodiments the nanoparticle is selected from the group consisting of a chromogenic nanoparticle, a semiconducting nanoparticle, a metallic nanoparticle, a ferromagnetic nanoparticle, a magnetic nanoparticle, an oxide nanoparticle, a fluorescent nanoparticle, a luminescent nanoparticle, a phosphorescent nanoparticle, an optically active nanoparticle and a radioactive nanoparticle.

According to an additional aspect of the present invention there is provided a method of delivering an active agent selected from the group consisting of a therapeutically active agent and a labeling agent, as described herein, to a bodily site of a subject in need thereof, the method comprising administering to the subject a composition which comprises the closed, hollow and self-assembled chemical multimer structure as described herein and such an active agent, the active agent being attached to and/or encapsulated in the chemical multimer structure.

According to further features in embodiments of the invention described below, the chemical multimer structure further comprises a targeting moiety attached thereto.

According to some embodiments the active agent is a therapeutically active agent and the associating groups in the chemical monomers forming the multimer structure are selected capable of forming a biocleavable bond.

According to some embodiments the active agent is a labeling agent and the associating groups in the chemical monomers forming the multimer structure are selected capable of forming a biostable bond.

According to still an additional aspect of the present invention there is provided a method of immunization comprising administering to a subject a composition which comprises the closed, hollow and self-assembled chemical multimer structure as described herein and a plurality of epitopes, the plurality of epitopes being attached to the chemical multimer structure.

According to a further aspect of the present invention there is provided a plastic crystal comprising a plurality of the closed, hollow and self-assembled chemical multimer structure described herein.

According to further features in embodiments of the invention described below, the plastic crystal has a lattice selected from the group consisting of a 2-dimensional lattice and a 3-dimensional lattice.

According to some embodiments the plastic crystal further comprising at least one active agent being attached to and/or encapsulated in the chemical multimer structure.

According to some embodiments the at least one active agent is selected from the group consisting of a chemical compound and a nanoparticle.

According to some embodiments the plastic crystal further comprising a metal coating deposited over at least a portion of a surface of the multimer structure.

According to still a further aspect of the present invention there is provided a method of preparing a composition which comprises the closed, hollow and self-assembled chemical multimer structure described herein and an active agent being encapsulated in the chemical multimer structure, the method comprising: (a) providing the plurality of chemical monomers that form the self-assembled chemical multimer structure, wherein each of the chemical monomers comprises a structurally symmetric core and a plurality of at least one type of associating groups, the plurality of associating groups being symmetrically positioned at a periphery of the structurally symmetric core, whereas the chemical monomers have structural complementarity to one another so as to form the closed, hollow and self-assembled chemical multimer structure upon occurrence of an associative proximity and orientation of the associating groups; and (b) subjecting the plurality of the chemical monomers to conditions allowing the chemical monomers to associate therebetween via the associating groups in the presence of the active agent, thereby creating the closed, hollow and self-assembled chemical multimer structure having the active agent encapsulated therein.

According to yet a further aspect of the present invention there is provided another method of preparing a composition which comprises the closed, hollow and self-assembled chemical multimer structure described herein and an active agent attached to the chemical multimer structure, the method comprising: (a) providing the plurality of chemical monomers that form the self-assembled chemical multimer structure, wherein each of the chemical monomers comprises a structurally symmetric core and a plurality of at least one type of associating groups, the plurality of associating groups being symmetrically positioned at a periphery of the structurally symmetric core, whereas the chemical monomers have structural complementarity to one another so as to form the closed, hollow and self-assembled chemical multimer structure upon occurrence of an associative proximity and orientation of the associating groups; (b) attaching the active agent to at least one of the chemical monomers, to thereby obtain a plurality of the chemical monomers in which at least of the chemical monomers has the active agent attached thereto; and (c) subjecting the plurality of the chemical monomers in which at least of the chemical monomers has the active agent attached thereto to conditions allowing the chemical monomers to associate therebetween via the associating groups in the presence of the active agent, thereby creating the closed, hollow and self-assembled chemical multimer structure having the active agent attached thereto.

According to an additional aspect of the present invention there is provided a method of preparing a chemical monomer capable of forming a closed, hollow and self-assembled multimer structure having a dodecahedral morphology, the method comprising: providing a structurally symmetric core compound having a 5-fold rotational symmetry; symmetrically attaching at a periphery of the core compound a plurality of at least one type of associating groups, wherein the chemical monomer and the associating groups are selected such that the chemical monomer has a structural complementarity to identical and/or different chemical monomers, which allows forming the closed, hollow and self-assembled multimer structure upon occurrence of associative proximity and orientation of the associating groups.

According to yet an additional aspect of the present invention there is provided a compound having a structurally symmetric core having a 5-fold rotational symmetry, and a plurality of at least one type of associating groups symmetrically positioned at a periphery of the core.

According to a further aspect of the present invention there is provided a metal-coated chemical multimer structure comprising the closed, hollow and self-assembled chemical multimer structure of claim 19 and at least one metal deposited over at least a portion of the surface thereof.

According to further features in embodiments of the invention described below, the metal is selected from the group consisting of a conductive metal, a semi-conductive metal, a magnetic metal, a radioactive metal isotope and any alloy or mixture thereof.

According to some embodiments the metal is selected from the group consisting of silver, palladium, copper, gold, chromium, nickel, cobalt, iron, cadmium, platinum, uranium, iridium, zinc, manganese, vanadium, rhodium, ruthenium, mercury, arsenic, antimony, and any combination thereof.

The present invention successfully addresses the shortcomings of the presently known configurations by providing compounds (chemical monomers) and chemical multimer structures formed therefrom that have structural features that resemble viral capsids and hence can be beneficially utilized in a variety of pharmacological and material science applications.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the singular form “a”, “an” and “the” include plural references unless the context clearly dictates otherwise. For example, the term “a protein” or “at least one protein” may include a plurality of proteins, including mixtures thereof.

As used herein the term “about” refers to ±10%.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein throughout, the term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of some embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents the chemical structures of corannulene and exemplary compounds having an internal 5-fold symmetry and referred to as Compounds 1-7, each comprising a corannulene core and one type of associating groups attached at the corannulene periphery, and which can serve as chemical monomers for forming a self-assembled chemical multimer structure according to the present embodiments;

FIG. 2 presents a schematic illustration of three different monomer interspersion patterns for constructing a closed and hollow chemical hetero-multimer from four chemical hetero-monomer types having two associating group types, wherein each associating group is represented by a triangle colored red or yellow, and wherein the dashed lines connect two associating groups which are associate to form a bond in the resulting closed and hollow chemical hetero-multimer;

FIG. 3 presents a synthetic scheme of rudimentary syntheses for obtaining structurally symmetric compounds (chemical monomers), such as Compounds 1 and 5 (see, FIG. 1), which serve as chemical monomers according to the present embodiments;

FIG. 4 presents a synthetic scheme for preparing an exemplary structurally symmetric compound (a chemical monomer, Compound 3′) having five 5-methyl-γ-lactam moieties serving as associating groups according to the present embodiments;

FIG. 5 presents a synthetic scheme for preparing an exemplary chemical hetero-monomer (Compound 8) having four associating groups of one type and one associating group of another type, which can serve for forming a hetero-multimer structure, according to the present embodiments;

FIG. 6 presents four exemplary chemical monomers according to the present embodiments, each having a different set of associating groups, showing Compound 9 which exhibits hydroxamic acid associating groups around the edge of a corannulene, Compound 10 which exhibits trispyrazolylborate associative groups, Compound 11 which exhibits phenanthroline associating groups and Compound 12 which exhibits dithiocarbamate associating groups;

FIG. 7 presents a synthetic scheme of rudimentary syntheses for obtaining exemplary chemical monomers (e.g., Compounds 25 and 26) which can associate via disulfide bonds, s according to the present embodiments;

FIG. 8 presents a uni-scale size comparison between three different chemical spheroids, namely a fullerene (C₆₀) on the left, a self-assembled, closed and hollow chemical multimer structure in the middle, comprised of 12 copies of Compound 1, an exemplary chemical monomer according to the present embodiments, and a satellite tobacco mosaic virus (STMV), consisting of 60 identical copies of a single protein that make up the viral capsid (coating), on the right;

FIG. 9 presents an illustration of the hydrogen-bond network connecting the chemical monomers in a chemical multimer made of an exemplary chemical monomer (Compounds 13) having carboxylic groups as associating groups which form two hydrogen bonds between each pair of associating groups;

FIGS. 10a-c present an exemplary chemical monomer (Compound 1, sym-penta-γ-lactam-corannulene), with a schematic illustration of two different modes of hydrogen bond network configurations between three identical monomers (FIG. 10a), marked as Mode A on the left and Mode B on the right, a photograph of two physical and magnet-fitted model units of the monomer (FIG. 10b), wherein the model unit on the left-hand side of the photograph shows the concave face of the model unit and the magnets placed on the N-hydrogen and oxygen atoms of the γ-lactam, and the model unit on the right-hand side shows the convex face of the model unit and obscuring the magnets due to the curvature thereof, and a photograph of two physical models of dodecahedral hemispheres, each constructed from six identical magnet-fitted physical model units of sym-penta-γ-lactam-corannulene (FIG. 10c), wherein the hemisphere on the right-hand side of the photograph is built according to the hydrogen bond network configuration shown in Mode A, and wherein the hemisphere on the left-hand side of the photograph is built according to the hydrogen bond network configuration shown in Mode B;

FIG. 11 presents a schematic illustration of the two modes of hydrogen bond network configurations, referred to herein as Mode A and Mode B, that can associate an exemplary structurally symmetric compound, Compound 3, according to the present embodiments;

FIG. 12 presents a schematic illustration of the two modes of hydrogen bond network configurations, referred to herein as Mode A and Mode B, that can associate an exemplary structurally symmetric compound, Compound 5, according to the present embodiments;

FIG. 13 presents a schematic illustration of the two modes of hydrogen bond network configurations, referred to herein as Mode A and Mode B, that can associate an exemplary chemical monomer, Compound 6, according to the present embodiments;

FIG. 14 presents a schematic illustration of the two modes of hydrogen bond network configurations, referred to herein as Mode A and Mode B, that can associate an exemplary structurally symmetric chemical monomer, Compound 7, according to the present embodiments;

FIG. 15 presents a schematic illustration of the intricate hydrogen-bond network connecting three identical copies of two exemplary chemical monomers according to the present embodiments, Compounds 14-15, wherein each pair of associating groups form four parallel hydrogen bonds;

FIG. 16 presents an illustration of the assembly orientation of eight different exemplary chemical monomers according to the present embodiments, Compounds 16-23, which interact each with its identical counterparts via metal coordination binding;

FIG. 17 presents an illustration of the assembly orientation of an exemplary chemical monomer according to the present embodiments, Compound 24, which interacts each with its identical counterparts via electrostatic or salt-bridge bonds therebetween;

FIG. 18 presents an illustration of the assembly process of an exemplary chemical monomer according to the present embodiments, Compound 25, which interacts with its identical counterparts via binding of two sulfur atoms, each from a different monomer, so as to form disulfide bonds therebetween in the presence of sodium benzenethiolate;

FIGS. 19a-b present a schematic illustration of an exemplary combinatorial self-assembled hetero-multimer structure system using a library of 4⁵ (=1024) different chemical monomer types, each having a random combination of 4 possible associating groups marked by the letters W, X, Y and Z, wherein X is compatible for binding with Z, and W is compatible for binding with Y, both by forming three parallel hydrogen bonds therebetween (FIG. 19a), and a schematic illustration of the hydrogen bond network which forms in one example of a multimer structure which can assemble out of the combinatorial system, wherein the associating groups in the monomers are X and Z (FIG. 19b);

FIG. 20 presents a schematic illustration of a silver-coating process effected on a chemical multimer structure according to the present embodiments, using an exemplary functionalized chemical monomer having an aldehyde (5-pentanalyl) reactive moiety attached to each of the γ-lactam associating groups (Compound 27), which is allowed to form the multimer structure, followed by the addition of soluble silver ions, which leads to the formation of Intermediate 15 having nucleation of metallic silver on the surface of the multimer structure, followed further deposition of metallic silver under reducing condition, leading to the thickening of the metallic coating on the surface of the multimer structure and the formation of a spherical metallic nanoparticles;

FIG. 21 presents an exemplary functionalized chemical monomer, Compound 28, according to the present embodiments, having tetradentate chelating moieties (3-propyl-pentane-1,2,4,5-tetraamine or two ethylenediamine groups on a propyl) attached to each of its γ-lactam associating groups, which is useful for attaching a nucleating catalytic metal to the chemical multimer structure that form therefrom, so as to effect metallization of the same;

FIG. 22 presents a schematic illustration of a chemical monomer, according to the present embodiments, having an active agent (represented by a gray sphere) attached thereto, and the resulting multimer structure assembled therefrom having 12×5 (=60) active agent moieties attached thereto; and

FIG. 23 presents the structure of three exemplary structurally symmetric chemical monomers which can be generated from a common intermediate, 1,3,5,7,9-sym-pentaethynylcorannulene, showing that the three chemical monomers exhibit the same associating groups all around the periphery of a corannulene core, yet each monomer differs from the others by the size (length) of the associating group, wherein Compound 29 exhibits a —CO₂H carboxyl group, Compound 30 exhibits a propiolic acid carboxyl group, and Compound 31 exhibits a 4-ethynylbenzoic acid carboxyl group.

### DESCRIPTION OF EMBODIMENTS OF THE INVENTION

The present invention is of closed, hollow and self-assembled chemical multimer structures having dodecahedral morphology. More specifically, the present invention is of closed, hollow and self-assembled multimer chemical structures which are made of chemical monomers held together by means of associating groups, wherein each monomer has a structurally symmetric core having a 5-fold rotational symmetry and associating groups that are symmetrically positioned around the periphery of core. The present invention is further of methods of creating such chemical monomers and self-assembled chemical multimer structures made therefrom, and further of uses of the multimer structures as, for example, a drug delivery system, an immunization system, and in material science applications such as reproducible formation of nanoparticles.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As discussed hereinabove, one of the more challenging undertakings in modern chemistry is to provide the means to produce a consistently reproducible closed and hollow chemical structure which is further reversibly or irreversibly self-assembled from small and relatively simple chemical units, or monomers, and furthermore has the capacity to be manipulated chemically.

As further discussed hereinabove, one of nature's responsions for the need for a closed, hollow and reversibly self-assembled chemical structure, is the highly symmetric icosahedral viral capsid, which is composed of identical and symmetric capsomers (the subunits of the viral capsid), each made of a complex assembly of proteins that come together to form each capsomer.

A U.S. Provisional Patent Application, co-filed with U.S. Provisional Patent Application 60/906,899 and entitled “Self-assembled Polyhedra”, by the present assignee, which is incorporated by reference as if fully set forth herein, presents self-assembling multimeric physical models of closed polyhedral structures made of structurally symmetric subunits, which mimic, for example, the structure and self-assembly characteristics of naturally occurring viral capsids. This disclosure provides a demonstration of the concept of self-assembly of a closed and hollow icosahedral viral capsid from symmetric subunits in a scaled-up macroscopic physical model. The examples utilizing combinations of different subunits or “tile” types, provided in the U.S. Provisional Patent Application entitled “self-assembled polyhedra” (supra), describe how such self-assembly can go beyond homogeneous assemblies to create diverse closed and hollow polyhedral structures having more than one subunit type.

While conceiving the present invention, it was envisioned that the concept of self-assembly of a hollow icosahedral viral capsid from symmetric subunits, being 15 nm to 75 nm in diameter and having hundreds of thousands of atoms, can be scaled-down and implemented in a relatively small molecular structure being a few nanometers in diameter and made of a few hundred atoms.

While reducing the present invention to practice, several types of compounds having 5-fold symmetry were designed and studied for their capacity to be used as capsomers in a scaled-down capsid, or for forming other self-assembled chemical multimer structures having dodecahedral morphology.

Hence, according to one aspect of the present invention there is provided a compound which comprises a structurally symmetric core having a 5-fold rotational symmetry, and several associating groups of one or more types, which are symmetrically positioned around the periphery of the structurally symmetric core. Being designed for forming a chemical multimer structure, this compound is also referred to herein as a chemical monomer.

The term “symmetric”, as used herein, describes an object having subunits (constituents, herein “basic components”) that correspond to one another, relative to the whole object, via a symmetry operation or transformation; one subunit in a symmetric object is also referred to herein as “basic component”.

The term “symmetry”, as used herein, describes an exact correspondence of form and constituent (subunit) configuration about a center or an axis (rotation symmetry operation), on opposite sides of a point (inversion symmetry operation) or opposite sides of a dividing line or plane (reflection symmetry operation). In the context of the present embodiments, the term “symmetry” refers to point symmetry groups, which include rotations, reflections (point and plane inversions) and combinations thereof, and does not encompass infinite lattice groups, which also include axis and plane translations and glide reflections.

The phrase “symmetry operation” or “symmetry transformation”, as used herein, include: a rotation operation about an axis, a reflection operation through a plane and an inversion operation through a point. In some embodiments the present invention is directed at chemical entities; therefore the symmetry operation is a rotation operation about an axis which, unlike the reflection and inversion, does not invert the chirality of chemical entity.

Thus, the phrase “structurally symmetric”, as used herein, describes a structural feature of an object, and in the context of the invention, a structural feature of the core of the chemical monomer, which relates to its shape as having an internal symmetry, as defined hereinabove. For example, a cyclopropane molecule is structurally symmetric and has a 3-fold rotational symmetry, and a benzene molecule is structurally symmetric and has a 6-fold rotational symmetry. In the context of the present invention, the core of the chemical monomer is structurally symmetric and has a 5-fold rotational symmetry.

Exemplary structurally symmetric compounds that have a 5-fold rotational symmetry, and thus can serve as structurally symmetric cores for the compounds presented herein, include, without limitation, cyclopentadiene and corannulene.

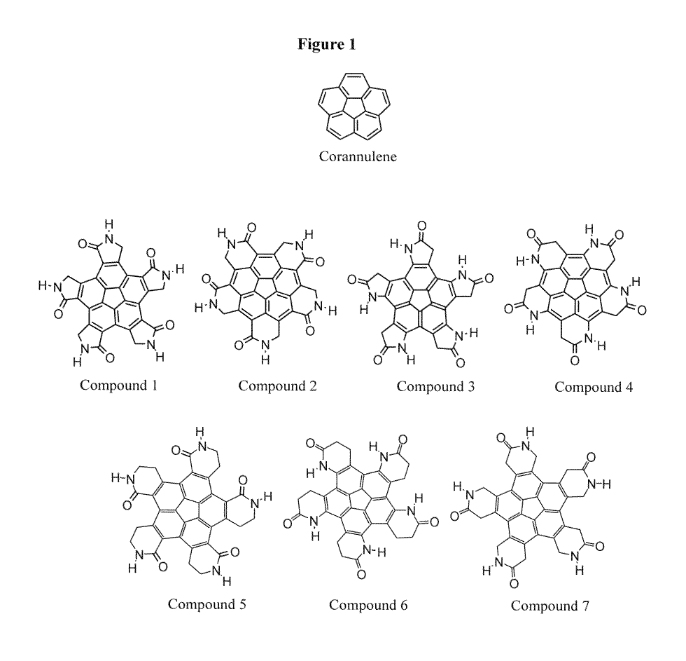

In one embodiment, the compound is based on corannulene (C₂₀H₁₀, see, FIG. 1), also known as the “buckybowl”. The multi-ring aromatic skeleton of corannulene is composed of a central pentagon which resembles a cyclopentadiene ring and 5 surrounding hexagons, each resembles a benzene ring. This rigid, 5-fold symmetric compound is also characterized by plain curvature, hence the alias buckybowl.

Cyclopentadiene-based compounds typically have a delocalized negative charge centered at the cyclopentadiene ring and thus often serve as ligands in organometallic complexes. Compounds having a structurally symmetric core which can further be utilized for forming coordinative complex with metals are beneficial in applications that involve metallized multimer structures, as discussed hereinbelow.

An exemplary cyclopentadiene-based compound which is suitable for serving as a chemical monomer according to the present embodiments is 4,4′,4″,4′″,4″″-(cyclopentadiene-1,2,3,4,5-pentayl)pentabenzamide, in which the benzamide substituents serve as associating groups of the type that can form, for example, hydrogen bonds with neighboring chemical monomers in a multimer structure. Alternatively the associating groups can be benzoic acid groups. Two such benzoic acid associating groups can form a benzoic acid anhydride, hence will tend to form labile (reversible) covalent bonds with neighboring chemical monomers in a multimer structure, which can be cleave readily under relatively mild conditions.

Other exemplary cyclopentadiene-based chemical monomers, having benzoic acid analog groups as associating groups, are illustrated in the general formula below:

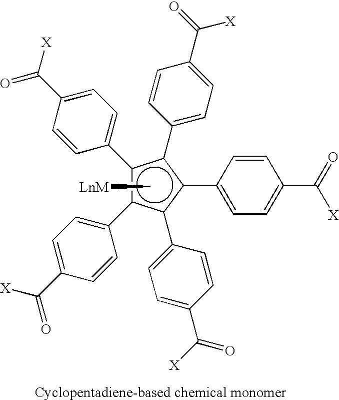

wherein X is, for example, OH, NH₂ or NHR′, and LnM represents a metal complex having n number of ligands coordinating a metal atom M at any acceptable valent (oxidation) state.

A compound forming the core of the chemical structure described herein typically lacks the ability to form large and well-ordered assemblies, thus the compound should comprise, other than the core which gives it the structurally symmetric skeleton, chemical moieties or associating groups which will endow the compound with the capacity to bind to other compounds. In some embodiments, the associating groups are distributed around the edge or periphery of the core such that the structural 5-fold symmetry is maintained in a gross manner, namely the associating groups are positioned according to the symmetry of the core but they are not necessarily the same associating groups all around the core.

In the context of the present embodiments, a compound as described herein, having a structurally symmetric core, may be a structurally symmetric compound (in cases where, for example, all associating groups around the 5-fold rotation axis of its core are the same), or, can be at least partially structurally symmetric compound (in cases where, for example, the associating groups around the 5-fold rotation axis of its core are different from one another). The compounds described herein are therefore also referred to as structurally symmetric compounds or structurally symmetric chemical monomers.

As used herein, the phrase “moiety” describes a part, and preferably a major part, of a chemical entity or compound, which typically has certain functionality or distinguishing features. A moiety being a major part of a chemical entity or compound typically represents herein that portion of the chemical entity or compound that is obtained upon covalently attaching the chemical entity or compound to another chemical entity or compound.

As used herein, the phrase “associating group” refers to a chemical group that can interact with another associating group so as to form a bond therebetween. The bond can be any type of chemical bond, including a combination of several types of bonds. Chemical bonds can be sorted to polar bond which have a dipole and thus are considered “directional” or polar, and non-polar bonds which lack a dipole or have a very weak dipole.

The terms “directional” and “polar”, as used herein, refer to a physical property of the interaction between two complementary yet different associating groups, as defined herein, which is characterized by a dipole having both a magnitude and a direction.

Directional or polar chemical bonds include, for example, hydrogen bonds, polar covalent bonds (between two different atoms or atoms which exhibit a difference in electronegativity), salt-bridges (electrostatic or ionic bonds), coordinative covalent bonds (dative bonds such as between a donor of an electron pairs and an acceptor and between a ligand and a metal ion), permanent-dipole to permanent-dipole bonds, and cation to aromatic bonds. Non-polar chemical bonds include, for example, covalent bonds (typically between identical atoms), hydrophobic interactions and aromatic interactions (pi-pi stacking).

As can be discerned from the definition of an associating group, the association occurs between at least two associating groups, each of which forms a part of a different compound (chemical monomer), and each of which can include a single atom or a group of atoms.

An example of a matching pair of associating groups which can form a hydrogen bond therebetween includes an oxygen atom and another oxygen atom, one as a hydrogen donor and another as a hydrogen acceptor, each being attached to a different molecule, and both share the same hydrogen atom which constitutes the hydrogen bond (e.g., a hydroxy group and a carbonyl). Similarly, a matching pair of associating groups can be an oxygen atom (such as in a carbonyl group) and a nitrogen atom (such as in an amine, amide or lactam group), two nitrogen atoms, and a fluorine atom and an oxygen or a nitrogen atom.

Hydrogen bonds are one of the most common chemical interactions in nature, and are responsible for the anomalous behaviors of water, the 3-dimensional structure of proteins and the complementary recognition and durability of nucleic acids (such as DNA and RNA), to name a few.

Similarly to nucleotide interactions, the associating groups described herein can be such that simultaneously form more than one hydrogen bond, while such a multiple hydrogen-bonds network can add to the strength of the interaction between these associating groups, as well as add to the specificity and structural complementarity between two compounds, as further discussed hereinbelow.

Exemplary associating groups which can simultaneously form more than one hydrogen bond include, without limitation, carboxyls, amides, lactams, imides and amidines, which can be used as associating groups per-se, or form a part of larger associating group such as pyrimidines (cytosine, thymine, uracil and the likes), purines (adenine, guanine, hypoxanthine, xanthine, theobromine, caffeine, uric acid and the likes) and other moieties that combine oxygen and nitrogen atoms (exemplary hydrogen bond donors and acceptors) as substituents attached to and/or incorporated in alkyls, aryls, heteroalicyclics and/or heteroaryls.

Another example of a matching pair of associating groups, which can form a ionic bond as another example of a polar bond, includes a pair of charged atoms or groups of atoms, one having a negative electrostatic charge and the other having a positive electrostatic charge, and each forming a part of a different compound. The charged atoms or group of atoms can be fully charged or partially charged, depending on the type of the associating group, the ionic character of the medium (solvent and solutes) and the pH of the medium in which the compound(s) is present. Exemplary negatively charged associating groups include carboxylates, sulfates, phosphates and the likes. Exemplary positively charged associating groups include ammonium, sulfonium, phosphonium and the likes.

Another example of a matching pair of associating groups, which can form a disulfide bond as an example of a non-polar covalent bond, includes a pair of sulfur atoms, each forming a part of a single compound. Compounds which include a thiol group can undergo disulfide bond formation therebetween when subjected to suitable oxidative conditions.

Another example of associating groups which interact via a third chemical entity includes at least a pair of electron-donating atoms (ligands) that form a coordinative bond with a metal ion. This type of bonding is oftentimes referred to as a metal-coordination complex, or an organometallic complex, wherein the metal coordination can occur by means of chelating moieties, where each of the associating groups comprises more than one donor atom, by a pair of single donor atoms, or a mixture thereof.

As used herein, the phrase “chelating moiety” describes a chemical moiety that is capable of forming a stable complex, such as an organometallic complex, with a metal, typically by donating electrons from certain electron-rich atoms present in the moiety to an electron-poor metal.

Chelating moieties typically contain one or more chelating groups. The phrase “metal-coordinating group”, also referred to herein and in the art as a “dentate”, describes a chemical group in the chelating moiety that contains a donor atom. The phrase “donor atom” describes an electron-rich atom that can donate a pair of electrons to the coordination sphere of the metal. Typical donor atoms include, for example, nitrogen, oxygen, sulfur and phosphor, each donating two (lone pair) electrons.

The chelating moiety is selected suitable for forming a stable complex with the desired metal. The stability of the metal-coordination complex typically depends on the number, type and spatial arrangement of the metal-coordinating groups surrounding the metal ion(s) and their fit to the coordination sphere of the metal.

Representative examples of metal-coordinating groups that may be included in chelating moieties according to the present embodiments therefore include, without limitation, amine, imine, carboxylate, beta-ketoenolate, thiocarboxyl, carbonyl, thiocarbonyl, hydroxyl, thiohydroxyl (thiol), hydrazine, oxime, phosphate, phosphite, phosphine, alkenyl, alkynyl, aryl, heteroaryl, nitrile, azide, alkoxy and sulfoxide.

A chelating moiety can be a monodentate chelating moiety, having one metal-coordinating group, a bidentate chelating moiety having two metal-coordinating groups, a tridentate chelating moiety having three metal-coordinating groups, a tetradentate chelating moiety having four metal-coordinating groups, or a chelating moiety having more than four metal-coordinating groups.

Thus, for example, the phrase “bidentate chelating moiety”, as used herein, describes a chelating moiety that contains two metal-coordinating groups linked one to the other (and hence provides two donor atoms), as described hereinabove, and thus can coordinatively bind two coordination sites of the metal. Representative examples of bidentate chelating moieties include, without limitation, hydroxamic acid, trispyrazolylborate, ethylenediamine, 2-mercapto-ethanol, 2-amino-ethanethiol, 3-amino-propan-1-ol, 2-amino-3-mercapto-propionic acid (cysteine), acetylacetonate, dithiocarbamate and phenanthroline.

The chelating moiety is selected suitable for forming a stable complex with the corresponding metal. The stability of the metal-coordination complex typically depends on the number, type and spatial arrangement of the metal-coordinating groups surrounding the metal ion(s) and their fit to the coordination sphere of the metal.

Thus, for example, metals such as cadmium, chromium, cobalt, copper, gold, iridium, iron, lead, magnesium, manganese, mercury, nickel, palladium, platinum, rhodium, ruthenium, iridium, silver, vanadium and/or zinc are known to form stable complexes with metal-coordination groups such as, for example, amine, imine, carboxyl, carbonyl, phosphine, nitrile and hydroxyl as well as with some nitrogen and oxygen containing heteroaryls such as furan, pyrrole, pyridine and phenanthroline. Examples of chelating moieties having such metal-coordinating groups and which can be utilized as metal-coordinating associating groups include, without limitation, iminodiacetate, ethylenediamine, diaminobutane, diethylenetriamine, triethylenetetraamine, bis(2-diphenylphosphinethyl)amine, and tris(2-diphenylphosphinethyl)amine.

Thus, for example, phenanthroline associating groups, form, in the presence of a metal cation such as Pd(II), Pt(II), Pt(IV), Ir(I) or Rh(III), either a square planar or an octahedral complex.

Similarly, metals such as mercury, arsenic, antimony and gold, are known to form stable complexes with metal-coordination groups such as amine, thiohydroxyl, hydroxyl, thiocarboxyl, thioalkoxy, thiosemicarbazide and thiocarbonyl. Examples of chelating moieties having such metal-coordinating associating groups and which can chelate these metals include, without limitation, dimercaprol, 2-mercapto-ethanol, 2-amino-ethanethiol, 3-amino-propan-1-ol, 2-amino-3-mercapto-propionic acid (cysteine), amidomercaptoacetyl, acetylacetonate and phenanthroline.

Correspondingly, transition metals such as techtenium and/or rhenium, optionally and preferably in the oxidized forms thereof oxorhenium(V) and oxotechnetium(V), are known to form stable complexes with metal-coordination groups such amine, oxime, hydrazine and thiol. In some embodiments these metals require a four metal-coordinating groups for optimal coordination, hence, exemplary complexes of oxorhenium(V) and oxotechnetium(V) typically include two bidentate chelating moieties in two associating groups, each attached to another compound.

The term “carboxyl”, as used herein, refers to a —C(═O)—O—R′, where R′ is, for example, hydrogen, alkyl, cycloalkyl, heteroalicyclic, aryl or heteroaryl, as these terms are defined herein. The term “carboxylate” refers to a negatively charged species of carboxyl, or —C(═O)O⁻—.

The term “alkyl” as used herein, describes a saturated, substituted or unsubstituted aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 5 carbon atoms. Stemming from the term alkyl are the terms cycloalkyl, alkenyl (alkene) and alkynyl.

The term “cycloalkyl” describes an all-carbon, substituted or unsubstituted monocyclic or fused ring (i.e., rings which share an adjacent pair of carbon atoms) group where one or more of the rings does not have a completely conjugated pi-electron system.

The term “alkenyl” refers to an alkyl group, as defined herein, which consists of at least two carbon atoms and at least one carbon-carbon double bond.

The term “alkynyl” refers to an alkyl group, as defined herein, which consists of at least two carbon atoms and at least one carbon-carbon triple bond.

The term “heteroalicyclic” describes a substituted or unsubstituted monocyclic or fused ring group having in the ring(s) one or more atoms such as nitrogen, oxygen and sulfur. The rings may also have one or more double bonds. However, the rings do not have a completely conjugated pi-electron system.

The term “aryl” describes an all-carbon monocyclic or fused-ring polycyclic (i.e., rings which share adjacent pairs of carbon atoms) groups having a completely conjugated pi-electron system.

The term “heteroaryl” describes a substituted or unsubstituted monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

As used herein, the term “amine” refers to an —NR′R″ group where R′ is as defined herein, and R″ is as defined herein for R′.

The term “amide” describes a —C(═O)—NR′R″, where R′ is as defined herein and R″ is as defined for R′.

The term “lactam”, as used herein, refers to a cyclic amide. Lactams typically have a prefixes which indicates the ring size, such as β-lactam (4-membered), γ-lactam (5-membered) and δ-lactam (6-membered ring).

As used herein the term “imide” refers to a —C(═NR′)—O—R″ group, where R′ and R″ are as defined herein.

As used herein the term “amidine” refers to a —C(═NR′)—NR″R′″ group, where R′ and R″ are as defined herein, and R′″ is as defined for R′.

As used herein, the term “sulfate” refers to a —O—S(═O)₂—O—R′, with R′ as defined herein, or to the negatively charged species thereof.

The term “phosphate” describes a —O—P(═O)(OR′)(OR″) group, with R′ and R″ as defined herein, or to the singly or doubly negatively charged species thereof.

The term “ammonium” as used herein refers to a positively charged amine, or a —N⁺R′R″R′″ group where R′, R″ and R′″ are as defined herein.

The term “sulfonium” as used herein, refers to a positively charged sulfur atom, or a —S⁺R′R″ group, where R′ and R″ are as defined herein.

The term “phosphonium” as used herein, refers to a positively charged phosphor atom, or a —P⁺R′R″R′″ group, where R′, R″ and R′″ are as defined herein.

The term “hydroxyl” describes a —OH group.

As used herein, the term “thiol” or “thiohydroxy” refers to a —SH group.

As used herein, the terms “carbonyl” as well as “acyl” refer to a —C(═O)-alkyl group, as defined hereinabove. A carbonyl can be a part of other chemical groups such as carboxylates and amides, as well as alpha-keto acids, or 2-oxo acids, such as pyruvic acid which have the carbonyl adjacent to the carboxylate; beta-keto acids, or 3-oxo acids, such as acetoacetic acid which have the carbonyl at the second carbon from the carboxylate; and gamma-keto acids, or 4-oxo acids, such as levulinic acid which have the carbonyl at the third carbon from the carboxylate.

The term “thiocarbonyl” as used herein, describes a —C(═S)-alkyl.

The term “imine”, which is also referred to in the art interchangeably as “Schiff-base”, describes a —N═CR′— group, with R′ as defined herein. As is well known in the art, Schiff bases are typically formed by reacting an aldehyde and an amine-containing moiety such as amine, hydrazine, hydrazide and the like, as these terms are defined herein.

As used herein, the term “beta-ketoenolate” refers to a derivative of a beta-keto acid, or a —R—C(═O)—CR′R″—C(═O)—R′″ group, where R′ and R″ are as defined herein, R and R′″ are as defined herein for R′ and R″.

As used herein, the term “thiocarboxyl” refers to an —C(═S)OR′ group, where R′ is as defined herein.

As used herein, the term “hydrazine” describes a —NR′—NR″R′″ group, where R′, R″ and R′″ are defined herein.

The term “hydrazide”, as used herein, refers to a —C(═O)—NR′—NR″R′″ group where R′, R″ and R′″ are defined herein.

The term “oxime” describes a ═N—OH group.

The term “phosphite” describes an —O—PR′(═O)(OR″) end group or an —O—PR′(═O)(O)— linking group, as these phrases are defined hereinabove, with R′ and R″ as defined herein.

The term “phosphine” describes a —PR′R″R′″ group, with R′, R″ and R′″ as defined herein.

The term “nitrile” or “cyano” describes a —C≡N group.

The term “isocyanate” describes a —N═C═O group.

The term “azide” describes a —N₃ group.

The term “alkoxy” as used herein describes an —O-alkyl, an —O-cycloalkyl, as defined hereinabove.

As used herein, the term “thioalkoxy” describes both a —S-alkyl, and a —S-cycloalkyl, as defined hereinabove.

The term “sulfoxide” or “sulfinyl” describes a —S(═O)R′ end group or an —S(═O)— linking group, as these phrases are defined hereinabove, where R′ is as defined hereinabove.

As used herein, the term “semicarbazide” refers to a —NR′—C(═O)—NR″—NR′″R* group, and the term “thiosemicarbazide” refers to a —NR′—C(═S)—NR″—NR″R* group, where R′, R″, R′″ and R* are define herein.

As used herein, the term “peroxo” refers to a —O—O—R′ group, where R′ is as defined hereinabove.

As used herein, the term “epoxide” describes a

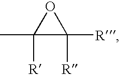

where R′, R″ and R′″ are as defined herein.

Each of the groups or moieties defined herein can be substituted by one or more substituents.

According to the present embodiments, a compound having a 5-fold symmetric core can have five associating groups attached thereto in a symmetric manner, such that, for example, all five associating groups are identical.

FIG. 1 presents an exemplary compound, sym-penta-γ-lactam-corannulene (see, Compound 1), in which all five associating groups are identical, hence defining the entire compound as structurally symmetric, possessing the appropriate structural complementarity to other identical monomers. The associating groups constitute five sets of symmetrically distributed hydrogen donors and acceptors which form a directional hydrogen-bond associative interaction with respect to other identical monomers.

FIG. 1 further presents the chemical structures of corannulene and of several exemplary structurally symmetric compounds, Compounds 2-7, which can serve as chemical monomers for forming a self-assembled chemical multimer structure. Each of Compounds 1-7 includes a corannulene core and lactam-based associating groups, yet each represents a different monomer- or tile-type by virtue of the way the lactam is attached at the corannulene periphery. The tiles presented in Compounds 1-7 are all internally symmetric, such that these compounds are structurally symmetric compounds and are further symmetric with respect to the associating groups, being all identical around the skeleton's 5-fold rotation axis.

Similar to some of the examples provided in U.S. Provisional Patent Application entitled “Self-assembled Polyhedra” (supra), which describes the use of a combination of different attachment entities (equivalent to the associating groups described herein) in one subunit or “tile” (equivalent to the present compound) that give rise to diverse polyhedral structures having more than one type of subunits, the compound presented herein can be designed to have more than one type of associating groups.

Thus, further according to the present embodiments, the compound may have more than one type of associating groups attached thereto. In this type of compounds, although the overall shape of the compound is grossly pentagonal by virtue of the symmetric core, the 5-fold symmetry of the compound is no longer maintained from the chemical aspect, and it is considered to be pseudo-symmetric from the structural aspect. An exemplary compound having two types of associating groups is Compound 8 which is illustrated below and further presented and discussed in the Examples section that follows.

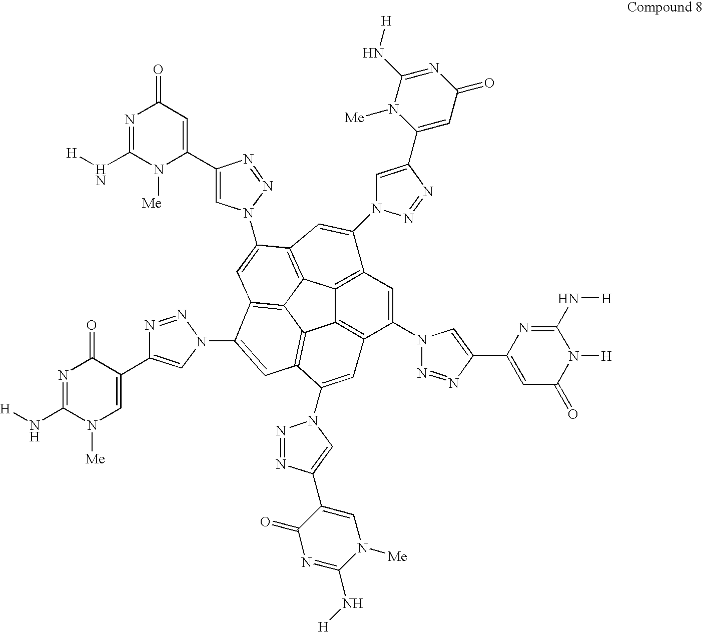

Compound 8 has five associating groups which are based on a pyrimidine derivative attached to a corannulene core via a triazole; each of the pyrimidine derivatives being capable of forming three parallel hydrogen bonds with a suitable counterpart associating group. Four of the associating groups comprise a pyrimidine derivative that presents two hydrogen acceptors and one hydrogen donor. The fifth associating group comprise a pyrimidine derivative that presents two hydrogen donors and one hydrogen acceptor. In the case of Compound 8, each of the types of associating group is incompatible to form three parallel hydrogen bonds with an identical associating group; however an associating group of one type can form three parallel hydrogen bonds with an associating group of another type.

In general, the associating groups can be selected so as to define the nature of a chemical multimer structure that is formed from the chemical monomers described herein, in terms of its reversible or irreversible assembly nature. Thus, the interactions between the associating groups can be of a reversible or irreversible chemical nature.

By “reversible” it is meant herein that the interactions between the associating groups can be de-activated under certain conditions, whereby such de-activation leads to disassembly of the multimeric structure. Such interactions include, for example, disulfide bonds, which can be decomposed in the presence of a reducing environment, hydrogen bonds, which can be decomposed by changing the pH of the environment, and biocleavable bonds, as defined herein, which can be decomposed under certain physiological conditions.

Thus, for example, when designing a chemical multimer structure which is capable of disassembling in a physiological environment, as detailed hereinbelow, the associating groups are selected so as to form a reversible bond therebetween, or otherwise a biocleavable bond.

As used herein, the phrase “biocleavable moiety” or “biocleavable bond” describes a chemical moiety or bond, which undergoes cleavage in a biological system by one or more cellular or extracellular component such as, for example, the digestive system of an organism or an enzymatic system in a living cell. Representative examples of biocleavable moieties include, without limitation, amides, carboxylates, carbamates, phosphates, hydrazides, thiohydrazides, disulfides, epoxides, and peroxides (peroxo). Such moieties are typically subjected to an enzymatic cleavage in a biological system, by enzymes such as, for example, hydrolases, amidases, kinases, peptidases, phospholipases, lipases, proteases, esterases, epoxide hydrolases, nitrilases, glycosidases and other cellular components.

By “irreversible” it is meant that the interactions between the associating groups are not readily de-activated and hence the multimer structure should be subjected to harsh conditions in order to disassemble. Such interactions include, for example, stable covalent bonds. An example of irreversible chemical interactions between the associating groups is a biostable bond, which typically remain intact under physiological conditions.

The compounds presented herein may have at least one additional reactive moiety attached to the core and/or to any one of the associating groups, which can be utilized for attaching thereto various substances and agents, as discussed in detail hereinbelow. In some embodiments, the reactive moiety forms a part of the associating group in such a way that the association of the associating groups is not interrupted by the presence of the reactive moiety and vice versa, the reactive group is not affected by the assembling process.

The phrase “reactive moiety”, as used herein, describes a chemical group that is capable of undergoing a chemical reaction that typically leads to a bond formation. The bond can be a hydrogen bond, an ionic bond, a coordinative bond, and, in some embodiments is a covalent bond. Chemical reactions that lead to a bond formation include, for example, nucleophilic and electrophilic substitutions, nucleophilic and electrophilic addition reactions, alkylations, addition-elimination reactions, cycloaddition reactions, rearrangement reactions and any other known organic reactions that involve a reactive group.

In some embodiments, the reactive moieties are attached to the chemical monomer in such a configuration so as to allow them to point outwards with respect to the curvature of the chemical monomer. Such outwards configuration can be achieved, for example, by stereo-selective attachment of the reactive moieties to the chemical monomer. This outwards configuration will result in a chemical multimer structure having the reactive moieties on the exterior part of the structure.

According to some embodiments, exemplary reactive moieties, as defined hereinabove, include, without limitation, amide, amidine, amine, halide (halo), carboxyl, carboxyl, heteroalicyclic, heteroaryl, hydrazine, imide, imine, isocyanate, nitrile, epoxide, peroxo, and the likes.

The reactive group, when present in the compounds described herein, can be protected, namely, comprising a protecting group, or non-protected (free). Commonly utilized protecting group chemistry can be used for protecting the reactive groups described herein. The reactive group can be protected during the assembly process and be thereafter de-protected so as to be utilized to attach to the formed multimer structure another agent or substance.

The compounds described herein are designed to have the capacity to form self-assembled chemical multimer structures when allowed to interact with identical or similar compounds under conditions that favor the self-assembly process, hence each of the compounds is regarded and interchangeably referred to herein as a chemical monomer.

According to another aspect of the present invention, there is provided a process of preparing the chemical monomer described hereinabove, which is designed capable of forming a closed, hollow and self-assembled multimer structure having dodecahedral morphology. The process, according to this aspect of the present invention, is effected by providing a core compound having a 5-fold rotational symmetry, such as a corannulene, and attaching at least one type associating groups at a periphery of this core compound in a symmetric fashion.

Several exemplary chemical monomers are presented in the Examples section that follows along with the synthetic routes for their attainment. These chemical monomers are presented according to the type of chemical bond which is formed between their associating groups when the monomers are subjected to conditions which favor self-assembly thereof to a corresponding fully formed multimer structure.

According to another aspect of the present invention, there is provided a closed, hollow and self-assembled chemical multimer structure having a dodecahedral morphology, which is self-assembled from the chemical monomers presented and discussed herein, whereas these chemical monomers have structural complementarity to one another, thus forming the closed, hollow and self-assembled chemical multimer structure via associative proximity and orientation of their associating groups.

As used herein, the phrase “self-assembled” and its related noun “self-assembly”, refers to a fundamental principle which may start from seemingly chaotic or random states and generates structural organization on all scales from molecules to large objects and systems, under stochastic conditions. In the context of the present invention, self-assembly is defined as a reversible or irreversible process in which pre-existing and disordered and randomly distributed compounds in a pre-defined system come together to form chemical structures having a pre-defined order. Examples of self-assembly include, without limitation, the formation of nano-scale to millimeter-scale layered structures lying in the interface between two liquids, the formation of a viral capsid from individual specific capsomers, and the formation of 2- and 3-dimensional lattices such as in crystals.

The term “closed” as used herein, is a relative term with respect to the size, the shape and the composition of two entities, namely an entity that defines an enclosure (the enclosing entity) and the entity that is being enclosed therein. In general, the term “closed” refers to a morphological state of an object which has discrete inner and outer surfaces which are substantially disconnected, wherein the inner surface constitutes the boundary of the enclosed area or space, which is secluded from the exterior area of space which is bounded only by the outer surface. Exemplary closed objects include, without limitations, an intact egg-shell, an intact and optionally inflated tire tube, a soap bubble and a C₆₀ buckminsterfullerene. In the context of the present invention, the closure of the enclosing entity depends of the size, shape and chemical composition of the entity that is being enclosed therein, such that the enclosing entity may be “closed” for one entity and at the same time be “open” for another entity. For example, a semipermeable membrane, such as commonly used for filtration and selective dialysis, may be shaped into a closed entity, such as a dialysis bag. The molecular weight, which corresponds to the size of each of the molecular species which are put in the dialysis bag, will determine if the dialysis bag is closed or not closed per each molecular species. Hence the molecular weight above which the molecular species cannot pass through the membrane is referred to as the molecular weight “cut-off” value of the membrane. Thus, in the example of a dialysis bag, a typical selective dialysis membrane having a given molecular weight “cut-off” which is shaped into a bag is not-closed for a water molecule, while it is closed for a protein molecule having a molecular weight above the “cut-off” value. Similarly, the closed, hollow and self-assembled chemical multimer structures presented herein are closed with respect to certain chemical entities which cannot pass through their enclosing shell, while the same multimer structures are not closed with respect to other entities. For example, the multimer structures of the present embodiments may be closed with respect to, for example, a drug molecule, but not-closed with respect to, for example, a single atom ion or an atom of a noble gas.

The term “hollow”, as used herein, refers to an object having a vacuous cavity, a gap or an empty space enclosed within. The hollowness or emptiness, in the context of the present invention, is a relative term which refers to the entities that can displace the hollowness and fill the enclosed space, therefore the enclosed space can be filled with displaceable substance and does not necessarily refers to vacuum or lack of substance.

The term “multimer” as used herein refers to an object which consists of a finite number of units, also referred to as monomers. In the context of the present invention, the closed, hollow and self-assembled chemical structure is a multimer of chemical monomers, each of which can be described as a single member of a set of similar or identical sub-units of a multimer structure.

The phrase “dodecahedral morphology” as used herein encompasses a chemical multimer structure which has true dodecahedral symmetry, as defined herein, as well as a chemical multimer structure that has apparent dodecahedral symmetry and/or pseudo dodecahedral symmetry, namely, a chemical multimer structure that mimics, yet is not, a true dodecahedron. It is noted herein that a regular dodecahedron is a dual polyhedron of another polyhedron known as the icosahedron, and both polyhedra possess icosahedral symmetry.

The phrase “structural complementarity” refers to a three-dimensional structural feature of the accessible surface of a given compound or chemical entity, or an assembly of such compounds or entities. Structural complementarity forms the means to molecular recognition that allows the structure of one molecule to interact with another molecule similarly to matching pieces in a puzzle, to splines or tenons dovetailed into their corresponding grooves in a machine, or to a lock and its corresponding key. The phrase “structural complementarity” is meant to encompass chemical compatibility as well as spatial compatibility. Hence compounds in general, by virtue of their architectural and functional diversity, can interact with one another based on structural complementarity which combines (i) a suitable and matching placement or positioning of one or more chemically-corresponding associating groups which are selected as members of a binding pair that can form one or more bonds with their corresponding member of the binding pair, and (ii) the overall structural features of the compound which depend mostly on the core structure of the molecule to which the associating groups are attached.

The compounds described hereinabove, which have 5 identical associating groups, are also referred to as homo-monomers, and can be used to form a homo-multimer structure, composed of identical chemical monomers. The compound described hereinabove, which include two or more types of associating groups attached thereto, are also referred to herein as hetero-monomers, and can be used to form hetero-multimer structures.

Compounds which include two or more types of associating groups attached to the core can comprise, for example, directional associating groups that form directional bonds, such as a donor-acceptor pair of a hydrogen bond, that are found in the compound at a ratio of 1-to-4 or 2-to-3. The selection and type of associating groups can be more complex, as in the case of Compound 8 described hereinabove and presented in the Examples section that follows. The use of such hetero-monomers will give rise to diverse hetero-multimer structures.

For example, a chemical monomer which has two types of associating groups wherein each type of associating group is less compatible for binding with its own kind and more compatible for binding with another, as, for example, in the case of Compound 8, would require different types of chemical monomers in order to self-assemble into one chemical hetero-multimer structure from a mixed set of 12 monomers. In such a case, each type of monomer should possess a different ratio of the two associating groups types attached thereto, and be present at a particular frequency (X out of 12) with respect to the other monomer types. Specifically, one such hetero-multimer structure comprises (i) 5 hetero-monomers (a frequency of 5 out of 12) which have 5 associating groups at a ratio of 1-to-4 with respect to their type; (ii) 5 hetero-monomers (a frequency of 5 out of 12) having associating groups at a ratio of 4-to-1 with respect to their type (an inverted ratio with respect to the previous monomer type); 1 homo-monomer (a frequency of 1 out of 12) which has a ratio of associating groups of 5-to-0, meaning 5 identical associating groups; and 1 homo-monomer which has a ratio of associating groups of 0- to-5, meaning 5 identical associating groups of the other type with respect to the previous monomer.

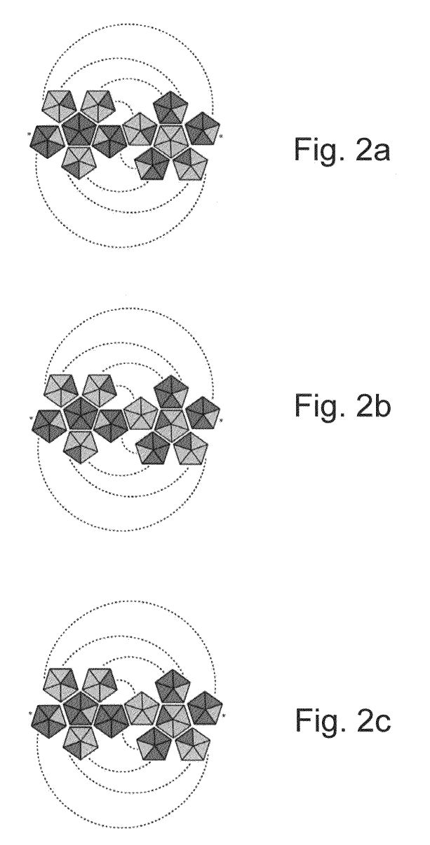

A diverse set of three hetero-multimer structures can be formed from the monomer populations described above, each having a different monomer interspersion patterns or combinatorial schemes, as illustrated schematically in FIG. 2. FIG. 2 presents a schematic illustration of three combinatorial schemes for constructing a closed and hollow chemical hetero-multimer from four monomer types having two different associating group types, wherein each associating group is colored red or yellow, and wherein the dashed lines connect two associating groups that associate to form a bond in the resulting closed and hollow chemical hetero-multimer. As can be seen in FIGS. 2a-c, this system of four monomer types can be arranged so as to form a closed and hollow chemical hetero-multimer in three different monomer interspersion patterns.

According to another aspect of the present invention, there is provided a method of creating the closed, hollow and self-assembled chemical multimer structure presented and discussed hereinabove, which is generally effected by:

providing numerous chemical monomers that form the self-assembled chemical multimer structure; and subjecting the chemical monomers to conditions which allow them to associate therebetween via the associating groups.

As presented hereinabove, each of the chemical monomers comprises a structurally symmetric core having a 5-fold rotational symmetry, and several associating groups of one type or more. The associating groups are symmetrically positioned at the periphery of the symmetric core, such that the chemical monomers have structural complementarity to one another and thus are capable of forming the closed, hollow and self-assembled chemical multimer structure when allowed to interact therebetween. The chemical monomers self-assemble into the chemical multimer structure upon occurrence of an associative proximity and orientation of associating groups on different chemical monomers, which allows the formation of bonds therebetween;

The self-assembly process is based on a series of associative chemical reactions between at least two chemical monomers, which occurs when the associating groups on one chemical monomer are in sufficient proximity and are oriented so as to allow constructive association with another chemical monomer. In other words, an associative interaction means an encounter that leads to the formation of a bond, as described herein, between two associating groups of at least two chemical monomers which results in the attachment of the chemical monomers to one another.

As in all associative chemical reactions, the formation of a bond between two compounds also depends on sufficient proximity and relative orientation between the compounds, and particularly between the associating groups within the compounds. In the context of the present embodiments, the degree of sufficient proximity depends on the attractive forces that can be exerted by the associating groups and the relative reactivity thereof.

The phrase “attractive force”, as used herein, refers to physical forces that span and have an effect over a distance, or field, such as electric and magnetic fields. Associating groups which can exert an attractive force field may attract each other over a definable distance, such as in the case of atoms having electrostatic charges.

The term “proximity” as used herein therefore describes any distance that allows interaction between such associating groups, whereby this distance can be practically null and depends on the presence, type and extent of the attractive forces which can be exerted by and affect the associating groups.

A pair of associating groups on two chemical monomers should also be oriented appropriately so as to allow a constructive encounter therebetween which results in the formation of a chemical bond. This is particularly important in cases where the associating groups are characterized by radial asymmetry, directivity, polarity, dipole, vectorial force, effective angle and/or other directional and spatial characteristics. An appropriate orientation is determined by steric constrains, surface accessibility and other structural complementarity considerations as described hereinabove. The term “orientation” therefore refers to a steric location and directionality of an object with reference to another object (herein the associating groups).

Regardless if the associating groups exert an attractive force field which extends beyond the physical boundary of the chemical monomer or not, or the degree of mutual reactivity of the associating groups, the monomer(s) must be subjected to suitable conditions which will allow them to associate therebetween. By suitable conditions it is meant that the chemical monomers need to be present at an adequate density (concentration) and possess suitable kinetic energy (temperature) so as to produce a sufficient number of events in which the monomers come in contact in the chemical sense, interact and associate (joined together). By “interact” it is meant that one or more monomer(s), each having associating groups thereon, while being subjected to suitable conditions as discussed hereinbelow, can come close enough to one another, and at a certain angle range, so as to allow the associating groups to be attached to one another.

In addition to an adequate concentration and suitable temperature, the condition which allow the self-assembly of a chemical multimer structure include other factors which affect the chemical environment in which the monomers are placed. These factors include the type of medium (solvent), the ionic strength and pH of the medium (solutes and buffers) and the presence of other chemical agents such as catalysts, oxidation and reduction agents, and other factors which affect the reactivity of the associating groups.

Closed, hollow and self-assembled chemical structures having dodecahedral morphology, as described herein, can be used in a myriad of applications, owing to several of the following most consistent and unique characteristics:
a capacity to assemble and optionally disassemble under particular chemical and physical conditions;
a hollow and closed interior;
an ability to be functionalized readily, as detailed hereinbelow;
a uniform and reproducible distribution of shape, size and composition;
a spherical overall shape; and
a wide range of controllable sizes.

One of the most intuitive uses of a closed and hollow molecular sphere that can reversibly self-assemble is a vehicle for substance retention, and subsequent release thereof in or to a chemical and/or biological system. Other uses of the closed and hollow multimer structures described herein may utilize the unique structural features of these chemical multimers delineated hereinabove.

Hence, according to another aspect of the present invention, there is provided a method of preparing a composition which comprises the closed, hollow and self-assembled chemical multimer structure presented hereinabove, and an active agent or otherwise a substance being encapsulated in the chemical multimer structure. The method, according to this aspect of the present invention, is generally effected by:

providing chemical monomers as described hereinabove; and subjecting these chemical monomers to conditions that allow them to associate therebetween via their associating groups, in the presence of one or more active agents, as these are defined and discussed hereinbelow, so as to form the closed, hollow and self-assembled chemical multimer structure around the active agent(s). Using this method, a closed, hollow and self-assembled chemical multimer structure in which the active agent(s) are encapsulated, is obtained.

The terms “encapsulate” and/or “entrap” and their grammatical diversions and conjugations, as used in the context of the present embodiments, relate to any form of accommodating a substance, herein the active agent, within a closed and hollow structure, herein the multimer structure. In some embodiments, entrapment of the active agent in the multimer structure, as in the context of the present embodiments, describes complete integration of the active agent within the multimer structure, such that the entrapped active agents are fully isolated from the surrounding environment as long as the multimer structure is assembled (closed).

As used herein, the terms “encapsulate” and/or “entrap” are meant to encompass cases where the encapsulated entity is solvated, e.g., the encapsulation includes solvent molecules. In cases where the encapsulated entity is surrounded by surface active agents, the encapsulation also includes the surrounding surface active agents.

The type of active agent which is suitable for encapsulation within a multimer structure according to the present embodiments depends on several characteristics thereof, such as its size, its solubility in the media in which the multimer structure is formed as well as other chemical compatibility criteria.

The encapsulation, according to the present embodiments, is meant to include the encapsulation of the solvent in which the encapsulation process takes place and/or the various solutes which are present in the solvent in addition to the chemical monomers and the active agent.

In cases where the active agent is not soluble under the conditions of the self-assembly process, the active agent can be solubilized by means of surface active molecules that surround the molecules of the active agent, which are encapsulated therewith in the encapsulation process.

The hollow void within a multimer structure wherein the active agent is encapsulated is set by the size of the core, the type of associating groups, and associating mode therebetween. Hence, the size of the hollow void within the multimer structure can be controlled by selecting suitable chemical monomers having particular associating groups.

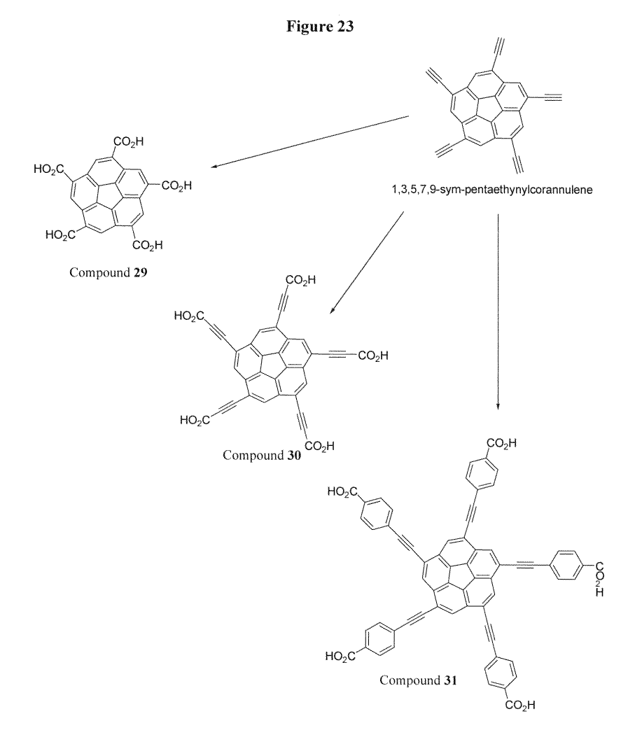

FIG. 23 presents three exemplary structurally symmetric chemical monomers which can be generated from a common intermediate, 1,3,5,7,9-sym-pentaethynylcorannulene. As can be seen in FIG. 23, all three chemical monomers exhibit the same associating group all around the periphery of the corannulene core, e.g., a carboxyl group, yet each monomer differs from the others by the size (length) of the associating group, wherein Compound 29 exhibits a —CO₂H carboxyl group, Compound 30 exhibits a propiolic acid carboxyl group, and Compound 31 exhibits a 4-ethynylbenzoic acid carboxyl group. Hence, a chemical multimer structure which is formed from Compound 29 will be smaller in diameter, and will have a smaller inner void as compared to a chemical multimer structure which is formed from Compound 30, and the latter will be smaller in diameter, and will have a smaller inner void as compared to a chemical multimer structure which is formed from Compound 31.

In cases where it is desirable to have the active agent(s) attached to the multimer structure, the active agent(s) can be attached to reactive moieties which form a part of the chemical monomers or multimer structure, as delineated hereinabove.

Hence, according to another aspect of the present invention, there is provided a method of preparing a composition which comprises the closed, hollow and self-assembled chemical multimer structure presented hereinabove, and an active agent attached to the chemical multimer structure.

The method is effected by:

(a) providing chemical monomers having at least one reactive moiety of at least one type attached thereto, as described in details hereinabove;

(b) attaching an active agent to at least one of said chemical monomers via a suitable reactive moiety, to thereby obtain a (functionalized) chemical monomer which has an active agent attached thereto, a process which is also referred to herein as functionalization; and

(c) subjecting these functionalized chemical monomers having the active agent attached thereto to conditions which allow them to associate therebetween via their associating groups, thereby creating the closed, hollow and self-assembled chemical multimer structure having active agents attached thereto.

Alternatively, a method of preparing a composition which comprises the closed, hollow and self-assembled chemical multimer structure presented hereinabove, and an active agent attached to the chemical multimer structure is effected by:

(a) providing chemical monomers having at least one reactive moiety of at least one type attached thereto, as described in details hereinabove;

(b) subjecting these chemical monomers having reactive moieties attached thereto to conditions which allow them to associate therebetween via their associating groups, so as to form a (functionalized) closed, hollow and self-assembled chemical multimer structure having reactive moieties attached thereto; and

(c) attaching an active agent to said (functionalized) chemical multimer structure via the reactive moieties.

Further alternatively, any of the methods described herein for preparing a self-assembled multimer structure have an active agent attached thereto can be effected simultaneously with the method of forming a self-assembled multimer structure have an active agent encapsulated therein, to thereby obtain a closed, hollow, self-assembled chemical multimer structure having one active agent encapsulated therein and one active agent attached thereto.

In order to attach an active agent to the multimer structure, it has to be functionalized so as to be capable for conjugation with other substances.

The phrase “to be functionalized” or the term “functionalization”, as used herein, refers to the addition of functionality to a multimer structure. Functionality can be added in the form of one or more reactive moieties which form a part of each chemical monomer, or go further as to attach a moiety of an active agent to the multimer structure via such reactive moieties. The attachment or conjugation of an active agent or a moiety thereof to a multimer structure can take place prior of subsequent to the self-assembly process, i.e. the conjugation can be effected on the chemical monomers or on the fully formed multimer. The number and type of functionalities per multimer structure can be set at the stage of designing the monomer, and particularly in cases when the multimer comprises more than one type of monomer each having more than one type of associating groups, as presented hereinabove.

As mentioned hereinabove, the reactive moiety can from a part of the associating group. In this case, the number of types of reactive groups that can be added to any given multimer structure according to the present embodiments is related to the number of types of associating groups which exist in the given monomer or multimer. Hence, a monomer which has two types of associating groups can have in principle two types of reactive moieties attached thereto, and consequently two types of active agents moieties can be attached thereto.

The presence and type of a reactive moiety is considered when designing each particular monomer such that, for a non-limiting example, a monomer of one type can possess reactive moiety or moieties of one type, while another type of monomer will comprise another type of reactive moiety or moieties and a third type of monomer will not include any reactive moiety. According to this example, two types of active agent moieties can be attached to the multimeric structure at a predetermined ratio according to the frequency of appearance of each type of monomer in the multimer, as delineated hereinabove in the discussion regarding diverse multimer structures.

Hence, due to the number of different combinations of attachment options, namely the number of types of associating groups/reactive moieties, the number of types of monomers and the ability to conjugate the active agent moieties before the monomers are mixed together for the self-assembly, during the formation of the multimer and/or after the multimer is formed, there is a great number of ways one or more types active agent moieties can be attached to or be encapsulated in one multimer structure.

Chemical multimer structures having one or more active agents attached thereto and/or encapsulated therein can be used to form compositions which can be utilized in many medicinal, analytical and mechanical uses.

Hence, according to another aspect of the present invention, there is provided a composition comprising the closed, hollow and self-assembled chemical multimer structure presented herein and at least one active agent being encapsulated in and/or attached to the chemical multimer structure.

As used herein, the phrase “active agent” refers to a compound or a portion of a compound, that exhibits a specific activity such as, for example, a physicochemical activity (e.g., spectral-, electronic-, magnetic-, conducting/semiconducting activity and the likes, as discussed hereinbelow), surface-modification activity (e.g., solubility, hydrophobicity, hydrophilicity, lubrication, bioavailability and protection), therapeutic activity (by means of a therapeutically active agent), targeting activity (by means of a targeting agent), labeling activity (by means of a labeling agent, e.g., for imaging and diagnostic purposes) and more.

As used herein, the phrase “pharmaceutically active agent” describes a chemical substance, which exhibits a beneficial activity when administered to a subject and includes, for example, a therapeutically active agent and a labeling agent.

As used herein, the phrase “therapeutically active agent” describes a chemical substance, which exhibits a therapeutic activity when administered to a subject.

Non-limiting examples of therapeutically active agents that can be beneficially utilized in this and other contexts of the present invention include, without limitation, a drug, a chemotherapeutic agent, an amino acid, a peptide, a polypeptide, a protein, an antigen, an antibody, a nucleic acid, a nucleic acid construct, a gene, a cardiovascular agent, a co-factor, a cytokine, a growth factor, a heparin, a hormone, a ligand, a lipid, a metabolite, a phospholipid, a prostaglandin, a receptor agonist, a receptor antagonist, a toxin, a vitamin, an agonist, an analgesic, an antagonist, an antibiotic, an antidepressant, an anti-diabetic agent, an anti-histamine, an anti-hypertensive agent, an anti-inflammatory drug, an anti-metabolic agent, an antimicrobial agent, an antioxidant, an anti-platelet agent, an anti-proliferative agent, an anti-psychotic agent, an antisense, an anti-thrombogenic agent, an enzyme, an epitope, an immunoglobulin, an inhibitor, an oligonucleotide, and any combination thereof.

As used herein, the term “targeting agent” describes agents which have a specific affinity to a desired bodily site (e.g., particular organ, cells and/or tissues). Targeting agents may be used to deliver a multimer structure to which they are attached to the desired site. The result is an enhanced effect and an improved exposure of a targeted organ, cells and/or tissues to any (other) agent conjugated to or encapsulated in the same multimer structure. In an exemplary embodiment, the targeting agent comprises a cell-internalizing moiety, such that the multimer structure can be utilized in gene therapy.

Exemplary targeting agents include, without limitation, porphyrins, hormones, antibodies and fragments thereof, and receptor ligands which bind to receptors that are expressed at specific bodily sites. Exemplary cell-internalizing moieties include, without limitation, positively charges (at physiological environment) moieties such as guanidines and amines, and moieties containing same (e.g., arginine and lysine).

As used herein, the phrase “labeling agent” refers to a detectable moiety or a probe and includes, for example, chromophores, fluorescent agents, phosphorescent agents, heavy metal clusters, and radioactive labeling agents, as well as any other known detectable agents.

As used herein, the term “chromophore” refers to a chemical moiety that, when attached to another molecule, renders the latter colored and thus visible when various spectrophotometric measurements are applied.

The phrase “fluorescent agent” refers to a compound that emits light at a specific wavelength during exposure to radiation from an external source.

The phrase “phosphorescent agent” refers to a compound emitting light without appreciable heat or external excitation as by slow oxidation of phosphorus.

A heavy metal cluster can be for example a cluster of gold atoms used, for example, for labeling in electron microscopy techniques.

Attaching one or more labeling agents to the multimer structure, allows, for example, detection of the multimer structure upon its administration, by applying a detection technique which is appropriate for a given labeling agent. For example, when the labeling agent is an imaging agent, the appropriate detection technique is an imaging technique. Using such imaging technique on a labeled multimer structure that is administered to a subject allows monitoring its distribution in the subject. In cases where mapping (imaging) the location of particular cells and/or tissues may be critical for the diagnosis and efficient treatment of a disease or medical condition, the labeled multimer structure can have an additional targeting moiety attached thereto which has a strong and specific affinity to those particular cells and/or tissues in question.

As used herein, the phrase “imaging agent” is meant to refer to agents which emit a detectable signal which can be traced to a particular position coordinates in the subject's body, wherein a full or partial signal detection scan can produce a set of coordinates that can be converted into an image showing the location(s) of the imaging agent(s) in the subject's body.

According to some embodiments, one multimer structure can have more than one active agents attached thereto and/or encapsulated therein. This ability to select the type of active agent and the method it is associated (attached/entrapped) with the structure opens the way for many uses of compositions comprising the same.

Compositions comprising multimer structures which encapsulate and/or functionalized with one or more therapeutically active agent can be used for many medicinal purposes as pharmaceutical compositions.

For example, a composition which comprises a therapeutically active agent (e.g., a drug) attached to or encapsulated in a multimer structure as described herein, can be efficiently utilized for treating a medical condition that is treatable by the active agent. Such a composition can desirably further comprise a targeting moiety attached to the multimer structure, which enhances the affinity of the multimer structure to the desired bodily site where the therapeutic activity should be exerted (e.g., a specific organ, tissue or cells).

While the therapeutically active agent can be encapsulated in or attached to the multimer structure, the targeting moiety is attached to the structure's exterior. In the case where the therapeutically active agent is encapsulated, the associating groups on the chemical monomers are selected such that they will dissociate in the body after a period of time and dissociate as soon as the chemical multimer is found in the physiological environment of the targeted cells tissue and/or organ. Alternatively, the therapeutically active agent can be attached to the multimer structure via a biocleavable bond, as defined hereinabove.

In one embodiment, compositions wherein the associating groups of the chemical monomers which form the multimer structure are selected capable of forming a biocleavable bond are beneficial for use in drug delivery.

One exemplary use of such a composition wherein specific drug delivery is crucial is a composition for gene therapy. Such a composition can include a multimer structure as presented herein and a combination of active agents attached to and/or encapsulated in a multimer structure. In order to design an effective tool for local gene therapy, a chemical multimer structure can have the following components:

a nucleic acid construct, an antisense or any other agent useful in gene therapy, for effecting the desired therapeutic effect within a cell, being attached to the multimer structure via a biocleavable moiety or being encapsulated in a multimer structure that can be disassembled under physiological conditions (e.g., being biocleavable by cellular components); and

a targeting agent attached to the multimer structure, selected to have an affinity to the desired location and optionally having a capacity for internalization into the cells at the desired location.

In this exemplary composition the multimer structure acts as a temporary transport vehicle for the therapeutic agent, which is driven to the targeted cells by virtue of the affinity of the targeting moieties attached thereto. Once reaching its designed target, the composite multimer structure is internalized into the cells owing to the preferred characteristic of the targeting moiety, and once inside the cell, the therapeutic agent is released upon interaction with cellular components that cleave either the interactions within the multimer structure or the bond between the multimer structure and the active agent. Thus, the therapeutic agent is delivered to its final target and can exert its therapeutic activity.

In another example, the composition comprises a multimer structure as described herein and an anti-cancer drug attached thereto or encapsulated therein. Such a composition can further comprise a targeting moiety selected as having a high affinity to, for example, epidermal growth factor receptor that is over expressed in cancer cells, or to any other receptor that is over expressed in cancer cells. Such a composition is designed so as to release the active agent at the desired site, by means of using a biocleavable bond for attaching the active agent or by means of biocleavable bonds between the associating groups within the multimer structure, as discussed hereinabove.

Apart from having unique drug delivery attributes, as detailed herein, the multimer structures may include an active agent such as a labeling agent attached thereto. The conjugation of an additional active agent to a multimer structure having a labeling moiety attached thereto may afford an efficient imaging probe. When the additional active agent is a targeting moiety, the conjugation thereof to multimer structures having a labeling agent attached thereto, can assist in the location, diagnosis and targeting of specific loci in a host. When the additional active agent is a therapeutic agent, the conjugation thereof to multimer structures having a labeling agent attached thereto, can assist in monitoring the distribution thereof in the host.

The use of the chemical multimer structure as a delivery vehicle depends largely on its capacity to penetrate at least some physiological barriers and biologic degradation. To this effect, the chemical multimer structure can be functionalized so as to alter its surface for higher biocompatibility. Such alteration can improve the bioavailability of any other active agents attached to and/or encapsulated in the multimer structure.

Exemplary surface-active agents that can provide such bioavailability include certain polymers, which are know to exert the desired surface alteration to organic and non-organic entities.

The term “polymer” as used herein encompasses one or more of a polymer, a copolymer or a mixture thereof, as well as linear or branched form thereof when applicable.

Exemplary polymers for use in this context of the present invention should be desirable characterized as:

i) being capable of forming a covalent bond and a stable chemical interaction with the multimer structure via a reactive moiety, as defined hereinabove;

ii) being non-toxic, namely, the polymer and/or its metabolites have no harmful effects to a biological system upon administration;

iii) being highly soluble in aqueous solutions;

iv) being highly flexible so as to have the capacity to assume a wide range of conformations so as to have low immunogenicity and low rate of bio-degradation; and

v) being of suitable mass and size in order to endow a multimer structure sufficient mass and protection so as to avoid rapid clearance from the host.

These characteristics constitute some of the requirements which render a polymer suitable for conjugation with a chemical multimer structure, and in particular a therapeutic composition comprising the same. As discussed hereinabove, these requirements include endowing the chemical multimer structure with the necessary solubility and bioavailability, protection from clearance and degradation, masking from the immune system and extended half-life within the biological system it is administered to, and allowing the polymer to have favorable interaction with the chemical multimer structure.

The polymer is selected such that when it is attached to a chemical multimer structure:

a) the structure-polymer conjugate substantially preserves other characteristics of the non-conjugated chemical multimer structure;

b) the structure-polymer conjugate is substantially soluble in aqueous and physiological solutions such as saline, even and particularly when the non-conjugated structure is less soluble in the same solutions; and

c) the characterizing half life of the structure-polymer conjugate under physiological conditions is substantially greater than the half life of the non-conjugated chemical multimer structure under similar conditions.

Exemplary polymers which are suitable for conjugation with a chemical multimer structure according to the present embodiments include, without limitation, a polyalkylene glycol, a polyethylene glycol (PEG), a poly(lactic acid) (PLA), a polyester, a polyglycolide (PGA), a polycaprolactone (PCL), a polyamide, a polymethacrylamide, a polyvinyl alcohol, a polycarboxylate, a polyvinyl pyrrolidinone, a dextran, a cellulose, a chitosan, a hydroxyethyl starch (HES), and any copolymer thereof.

Polyethylene glycol (PEG) is a highly suitable polymer for conjugation with chemical multimer structures as presented herein and hence, according to some embodiments, the polymer is PEG. In some embodiments, the PEG moiety has an average molecular weigh that ranges from 1 kDa to 100 kDa.

Hence, according to some embodiments of the present invention, there is provided a chemical multimer structure and a polymer moiety covalently attached thereto. In some embodiments, the polymer is PEG, providing a PEGylated chemical multimer structure.

As exemplified hereinabove, the chemical multimer structures presented herein can be used as multipurpose delivery vehicle for a variety of active agents. Hence, according to another aspect of the present invention, there is provided a method of delivering an active agent such as a therapeutically active agent and/or a labeling agent to a bodily site of a subject in need thereof. The method is effected by administering to the subject a composition which includes the closed, hollow and self-assembled chemical multimer structure presented herein and one or more of the active agents being attached to and/or encapsulated in the chemical multimer structure.

Without being bound to any particular theory, is it assumed that the immune system has a greater sensitivity, and thus exerts a more intense response towards spherical objects which present epitopes on its surface which perturb into the media. Hence, attaching epitopes to a chemical multimer as presented herein, should provide an effective tool for effecting controlled immunization in humans and animals.

In the context of the present embodiments, the term “epitope” refers to a part of a biological macromolecule (typically a protein, a polypeptide or a peptide) that evokes a response by the immune system of a subject, and more specifically recognized by antibodies, B cells or cytotoxic T cells of the subject. As used herein, epitopes are derived from non-self proteins.

To this effect, another example of a composition is designed for immunization and thus comprising a chemical multimer and one or more epitopes attached thereto such that the epitopes perturb outwards and are exposed to the environment so as to evoke an immune response when administered to a subject.

This exemplary composition, identified for use in immunization, includes a chemical multimer having attached thereto, for example, one or more peptides or protein segments taken from or representing a pathological microorganism or virus. Upon administration of this composition, the immune system of a subject will recognize the peptides or protein segments as non-self and an immune response towards these foreign agents will take place, but without causing any of the illness and other pathologic effects which are characteristic to the microorganism or virus.

Hence, according to another aspect of the present invention there is provided a method of immunization comprising administering to a subject a composition which includes the closed, hollow and self-assembled chemical multimer structure presented herein having a plurality of epitopes attached to surface of the chemical multimer structure.

Attachment of an active agent to the chemical multimer structures presented herein can further be effected so as to modify the surface of the structure, when utilized in non-medicinal applications. Thus, the active agent can be, for example, a hydrophobic, a hydrophilic or an amphiphilic substance.

The chemical multimer structures presented herein can further be utilized so as to enable or facilitate the analysis of chemical features of other chemical entities.

For example, the composition of a chemical multimer and an encapsulated substance (an active agent or any other chemical compound) can be used for analytical purposes, particularly when the analytical method is mass spectroscopy and when it is not possible to ionize the substance (analyte) or when the substance is otherwise non-ionized. Such a substance can be encapsulated in a multimer structure as described herein and the obtained composition is subjected to analysis. Thus, ionization for mass spectroscopy purposes is effected on the composition, and the resulting mass spectra is analyzed so as to retrieve the mass spectra of the analyte.

Another example includes a composition of a multimer structure having a chemical substance encapsulated therein, and the use thereof to form a “plastic crystal”, wherein the entrapped substance is held in a two- or three-dimensional lattice which is formed by the encapsulating multimer structures by virtue of their spherical and uniform shape. Such a unique ordered arrangement of a sample can be used to analyze the substance by technique which require an ordered sample, like X-ray crystallography and other light-scattering and diffraction techniques.

In another example, the entrapped entity is a nanoparticle or a plurality of nanoparticles encapsulated in a suitably sized multimer structure.

As used herein, the term “nanoparticle” describes one or more nano-sized discrete mass of solid particles being less than 0.01 micron in the largest axis thereof, and preferably being from about 1 nanometers (nm) to about 4 nm.

The nanoparticle can be, for example, a chromogenic nanoparticle, a semiconducting nanoparticle, a metallic nanoparticle, a ferromagnetic nanoparticle, a magnetic nanoparticle, an oxide nanoparticle, a fluorescent nanoparticle, a luminescent nanoparticle, a phosphorescent nanoparticle, an optically active nanoparticle and a radioactive nanoparticles, and therefore the composition of the multimer structure having the above nanoparticles will substantially be characterized according to the chemical and physical properties of the encapsulated nanoparticles.

Nanoparticles can be categorized by their crystallinity and hence can be crystalline nanoparticles (also known and referred to herein as nanocrystals), semi-crystalline nanoparticles or amorphous nanoparticles.

The term “crystalline” or “crystal” refers to a solid body bounded by natural plane faces that are the external expression of a regular internally ordered arrangement or lattice of constituent atoms, molecules, or ions.

The term “amorphous” as used herein refers to the lack of regular internally ordered arrangement, or the antithetical form of the crystalline form.

In some embodiments, the nanoparticles according to the present embodiments are nanocrystals.

The nanocrystals are generally members of a crystalline population having a narrow size distribution.

The nanoparticles entrapped in the multimer structures according to the present embodiments, and particularly nanocrystals, can be further sub-grouped by their properties.

The terms “semiconducting” and “semiconductive”, as used herein, refer to a characteristic of a solid material whose electrical conductivity at room temperature is between that of a conducting element and that of an insulating element. When exposed to heat, electric field or light of discrete wavelength, semiconductive nanoparticles change their electric conductivity from that of a conducting substance to that of an insulating substance and vice versa, depending on the type. In a semiconducting substance there is a limited movement of electrons, depending upon the crystal structure of the material constituting the substance. The incorporation of certain impurities in the lattice of a semiconducting substance enhances its conductive properties. The impurities either add free electrons or create holes (electron deficiencies) in the crystal structures of the host substances by attracting electrons. Thus, there are two types of semiconducting substances: the N-type (negative), in which the current carriers (electrons) are negative, and the P-type (positive), in which the positively charged holes move and carry the current. The process of adding these impurities is called doping; the impurities themselves are called dopants. Dopants that contribute mobile electrons are known as donor impurities; those that cause the formation of holes are known as acceptor impurities. Undoped semiconducting material is called intrinsic semiconductor material. Certain chemical compounds and elements, including, for example, silicone, gallium arsenide, indium antimonide, and aluminum phosphide are semiconducting elements. Semiconducting elements are often used to construct electronic devices such as diodes, transistors, and computer memory devices.

The phrase “magnetic” as used herein refers to a physical characteristic of a substance which exhibits itself by producing a magnetic field, thereby showing an aptitude to attract ferromagnetic substances, such as iron, and align in an external magnetic field. Magnetic nanoparticles in the context of the present invention, are nano-sized magnets, and can be utilized as such in applications which utilize this magnetic characteristic.

The phrase “optically active” as used herein refers to a characteristic of a substance which rotates the plane of incident linearly polarized light. The optically active nanoparticles according to embodiments of the present invention, include nanoparticles that rotate the electric field clockwise (dextrorotatory) and nanoparticles that produce a counterclockwise rotation (levorotatory), and are known as enantiomorphs. The optical activity of nanoparticles is typically associated with the crystal structure thereof, as evidenced by the fact that neither molten nor amorphous nanoparticles demonstrate optical activity.

The term “luminescent” refers to a characteristic of a substance that can emit all forms of cool light, i.e., light emitted by sources other than a hot, incandescent body. Luminescence is a collective term that is used to describe phenomena caused by the movement of electrons within a substance from higher energetic states to lower energetic states. There are many types of luminescence, including chemiluminescence, produced by certain chemical reactions at low temperatures, mainly oxidations, at low temperatures; electroluminescence, produced by electric discharges, which may appear, for example, when silk or fur is stroked or when adhesive surfaces are separated; and triboluminescence (illumination created through friction), typically produced by rubbing or crushing crystals. When the luminescence is caused by absorption of some form of radiant energy, such as ultraviolet radiation or X rays (or by some other forms of energy, such as mechanical pressure), and ceases as soon as (or very shortly after) the radiation causing it ceases, then it is known as fluorescence. If the luminescence continues after the radiation causing it has stopped, then it is known as phosphorescence.

As used herein, the term “chromogenic” refers to a physical characteristic of a substance that, when interacting with light of multiple wavelengths, discriminately absorbs, transmits and/or reflects light of specific wavelength(s) thus rendering the substance colored when visible and/or when various spectrophotometric measurements are applied. For example, dyes and pigments are chromogenic substances.

Exemplary semiconducting nanocrystals include, without limitation, InAs, CdS, Ge, Si, SiC, Se, CdSe, CdTe, ZnS, ZnSe, CdSe/ZnS or InAs/ZnSe core-shell nanocrystals. Exemplary metallic nanocrystals include, without limitation, Au, Cu, Pt, Ag and PbSe. Exemplary magnetic nanocrystals include, without limitation, Fe₂O₃, Co, Mn and the like.

The nanocrystals entrapped in the multimer structures can therefore be selected according to the desired application of the resulting composition, while exerting their unique characteristics from within the multimer structures.

Color-tunable nanoparticles are of great interest for various applications as inks, coatings, labeling and tagging, in optics, catalysis, sensing, in optical microcavities and as building blocks for photonic band-gap structures.

The compositions described herein can therefore be utilized, in addition to the provision of protected nanoparticles, to introduce optical, chemical and/or physical functionalities to the entrapping multimer structures, by taking advantage of the wide-range tunable absorption and emission, magnetic and/or radioactive characteristics provided by nanoparticles, and particularly by nanocrystals. Thus, the functional characteristic of the multimer structures of the present embodiments follows that of the nanoparticles, which bestow, for example, semi-conductivity, chromogenic activity, photo-electronic reactivity, optical activity, spectral activity, magnetism and radioactivity on the multimer structures, resulting in, for example, optically active, semi-conductive, chromogenic, magnetic and radioactive multimer structures.

The phase “chromogenic activity” describes phenomena which pertain to chromogenic characteristics of a substance, as these are defined herein. Chromogenic activity may be exhibited by the appearance of colors, typically in the visible range.

The phase “optical activity” refers to phenomena exhibited by optically active substances, as these are defined herein.

The phase “spectral activity” as used herein refers jointly to chromogenic, fluorescent, phosphorescent, luminescent and other optical activities, as these are defined herein.

The phase “semi-conductivity” refers to phenomena exhibited by semiconducting substances, as these are defined herein.

The term “radioactivity” as used herein refers to the spontaneous emission of radiation, either directly from unstable atomic nuclei or as a consequence of a nuclear reaction. The radiation emitted by a radioactive substance, includes alpha particles, nucleons, electrons, positrons and gamma rays.

The phase “photoelectronic reactivity” as used herein refers jointly to semiconductivity, spectral activity and the phenomena known as photoelectric effect. The photoelectric effect is expressed by the ejection of electrons from a substance caused by incident electromagnetic radiation, especially by visible light.

The phase “magnetism” refers to phenomena exhibited by magnetic, substances, as these are defined herein.

Optically active and semi-conductive multimer structures as presented herein can be efficiently utilized in many applications such as, but not limited to, inks and paints, optical and photo-electronic labeling, optical filtration, electronic paper and barcoded tags.

Magnetic multimer structures as presented herein can be efficiently utilized in applications such as, but not limited to, magnetic liquids and fluids, magnetic separation and labeling of various cells, DNA/RNA fragments, proteins, small molecules and the likes.

Radioactive multimer structures as presented herein can be utilized in applications wherein tracing and detection of entities of interest is required, such as, but not limited to, chromatography, diagnostic and therapeutic nuclear medicine and the likes.

In order to bestow unique chemical and physical properties to the chemical multimers presented herein, the multimer structure can be functionalized so as to have the capacity to be coated with a layer of metal in its metallic (zero valent) state. A metal-coated (metallized) multimer structure can be regarded as a composite nanoparticle which is useful for all relevant intents and purposes as the abovementioned nanoparticles and other nanoparticles known in the art, by virtue of the thin yet significant metallic coat, and its reproducible nanometric size.

Well-defined and discrete metallized multimer structure nanoparticles are highly beneficial in terms of manipulation and the desired application of the nanoparticles. The spherical shape is ideal from various points of view, but mostly for the isotropism of emittance from, and absorption of energy into a globular object, and the ability to arrange spheres in tightly packed two-dimensional and three-dimensional lattices, namely, to cover a surface with a uniform film of one or more layers, and to fill in gaps and crevices, or be molded into any other larger shape. Therefore, most applications require a uniform shape and size so as to enable the utilization of predictable and desired chemical and physical characteristics of the nanoparticles.

Functionalization of multimer structures directed at metallization thereof can involve reactive groups, such as aldehydes and imines, which are either reductive in nature towards certain metals, such as silver, or capable of chelating specific metal atoms which serve as catalytic reduction sites for other metal atoms. As presented in the Examples section that follows, the chemical multimer structures presented herein can be metallized by well known electroless deposition processes which utilize these functionalization options.

In the case of nanoparticles which are composed of metallized multimer structures, the size, size distribution, uniformity, shape, discreteness and other properties of the metallized multimer structures can be finely controlled. The type of the deposited metal and the amount of metal (thickness of the layer) can be controlled by setting the appropriate chemical conditions for the electroless deposition process.

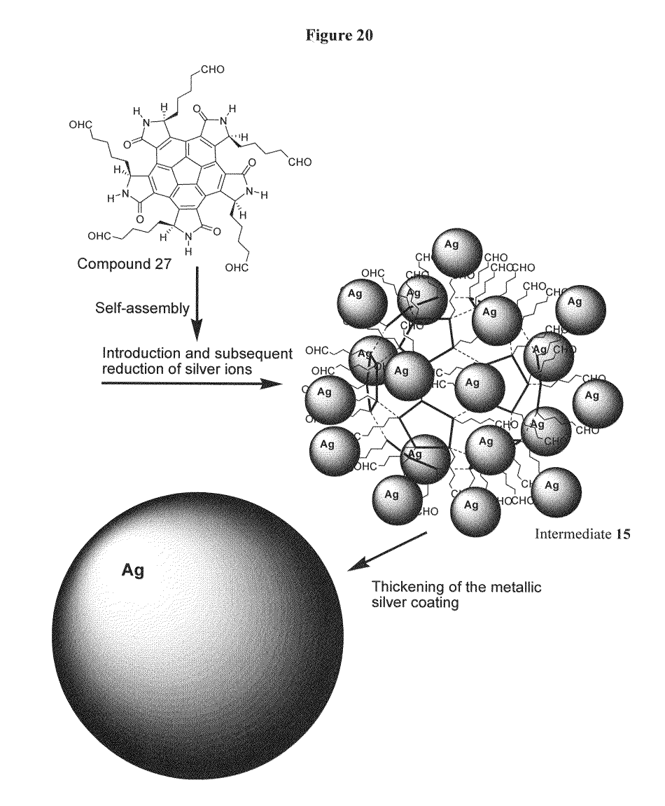

As presented in the Examples section that follows, in the specific example of a silver-metallized chemical multimer structure based on Compound 27 illustrated in FIG. 20, the outer diameter of the structure after the silver-coating process can reach a uniform diameter of about 4 nm which is an optimal size for most known applications of metallic nanoparticles. The uniform size of such metallic nanoparticles would allow their crystallization, thus generating a new form of matter, namely a crystalline solid that comprises spherical elementary unit in the form of metallized chemical multimer structure, each containing a pull of liquid in the interior thereof.

Such metallized multimeric structures, or composite nanoparticles, can be used, for example, in ultra-accurate mirrors, in molecular electronics, molecular computers and in any other application in which metallic nanoparticles are beneficial. Metallized multimeric structures that entrap a liquid can further be utilized.

A chemical multimer structure coated with a magnetic metal can be regarded as a magnetic nanoparticle and more specifically as a magnetic nanodot. A magnetic nanodot has north and south poles like a tiny bar magnet that can switch back and forth (or between a 0-state and a 1-state) in response to a strong magnetic field. Generally, the smaller the nanodot, the stronger the field required to induce the switch. Until now researchers have been unable to control the wide variation in nanodot switching response, owing to the design fault of the multilayer films that serve as the starting material for the nanodots. The magnetic metallized chemical multimer nanodots presented herein can overcome this major limitation by being formed according to processes that afford uniformly sized and uniformly coated and substantially smaller magnetic nanodots as compared to presently fabricated nanodots. The magnetic nanodots presented herein can be used, as a non-limiting example, in arrays that respond to magnetic fields with extremely high levels of uniformity. Such arrays of uniform magnetic chemical multimer metallized structures can be used in commercially viable nanodot drives having at least 100 times the data capacity of present commercially viable hard disk drives.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### Examples

Reference is now made to the following examples, which together with the above descriptions, illustrate the invention in a non limiting fashion.

### Example 1

### General Synthetic Pathway for Preparing Chemical Monomers for Forming a Self-Assembled Chemical Multimer (Dodecamer) Structure

In order to form a closed, hollow and self-assembled chemical multimer structure, for example dodecahedral multimer (dodecamer) chemical structure according to the present embodiments, the structural core of the chemical monomers forming the chemical multimer has to fulfill several requirements as follows:

=the structural core needs to possess an appropriate symmetry, curvature and rigidity so as to allow the self-assembly formation of the chemical multimer; and

=the structural core needs to be selected such that introduction of associating groups which, together with the symmetrical shape of the core provide the chemical complementarity on the surface of the structural core, can be performed.

Corannulene (C₂₀H₁₀, “buckybowl”), was selected as an exemplary compound that can serve as the structural core of the chemical monomers forming the self-assembled multimer chemical structure. The aromatic skeleton of corannulene, which is composed of a central pentagon and 5 surrounding hexagons, is a rigid, 5-fold symmetric compound with suitable curvature. Self-assembly of 12 copies of a corannulene-based compound (a “tile”) should produce, upon introduction of suitable, structurally and chemically complementing associating groups to each corannulene structure, a dodecahedral multimer chemical structure.

Thus, suitable associating group are introduced to each of the five edges of the corannulene core. The associating groups are selected so as to allow the self-assembly of the pentagonal corannulene-based tiles so as to form a dodecahedral multimer chemical structure via specific interactions between associating groups of neighboring tiles. These associating groups, can be identical all around the edges of the corannulene core, or different.

More specifically, the associating groups are selected such that they form a part of a binding pair which is a set of two compatible and complementary associating groups, both chemically and structurally. In some embodiments, the specific binding of such a pair is characterized by a high binding constant at specific chemical conditions, which yet, under other specific conditions, can optionally disassemble so as to result in disintegration of the chemical structure. The self-assembly and disassembly is one of the key features of these multimer structures, which render them highly suitable as, for example, efficient storage and delivery carriers.

The preparation of many polyaromatic structures comprising selective functionalization is performed according to any of several modern and efficient synthetic methodologies developed by, for example, Rabideau, Siegel and others [Lawrence T. Scott et al., Pure & Appl. Chem., Vol. 71, No. 2, pp. 209-219, 1999; Sygula A. and Rabideau P. W., J. Am. Chem. Soc., 122, p. 6323, 2000; Seiders T. J., Baldridge, K. K. and Siegel, J. S., Tetrahedron, 2001, pp. 3737-3742; and Ivan Aprahamian et al., J. Org. Chem., 2006, 71(1), pp. 290-298].

These methodologies can be utilized for the design and preparation of chemical monomers that comprise a corannulene, or any other structurally symmetric core and diverse associating groups that are symmetrically attached thereto. These associating groups can be categorized by the associative interactions therebetween, which lead to the formation of reversible bond and consequently to the assembly of a corresponding self-assembled multimer. Representative examples of such reversible bonds include hydrogen bonds, salt-bridge bonds, metal-ligand coordination bonds, hydrophobic interactions and a combination thereof.

Preparation of Tiles Which Interact Via Hydrogen Bonds:

As described above, each of the structurally symmetric compounds, or chemical monomers (tiles) presented herein has associating groups which are selected so as to enable binding with other compatible tiles via directional (polar) and optionally further reversible binding interactions, so as to form the closed, hollow and self-assembled chemical multimer structure. An exemplary associative interaction used to form the self-assembled chemical multimer is hydrogen bonding.

One exemplary group of chemical monomers includes compounds having a secondary amide associating group, which is an acceptor-donor pair, presented on each of the pentagon edges. An exemplary chemical monomer of that group, possessing the appropriate structural complementarity to other identical monomers is sym-pentam-γ-lactam-corannulene (see, Compound 1 in FIG. 1). This structural complementarity is stemming from 5 sets of symmetrically distributed hydrogen donors and acceptors which form a directional hydrogen-bond associative interaction between each monomer.

FIG. 1 presents the chemical structures of corannulene and of several exemplary structurally symmetric compounds, Compounds 1-7, which can serve as chemical monomers for forming a self-assembled chemical multimer structure. Each of Compounds 1-7 includes a corannulene core and represents a different tile-type by virtue of the way they are attached at the corannulene periphery. The tiles presented in Compounds 1-7 are all internally symmetric, such that these compounds are structurally symmetric compounds and are further symmetric with respect to the associating groups, being all identical around the skeleton's 5-fold rotation axis.

Compound 1 is a γ-lactam and Compound 2 is a δ-lactam isomer of Compound 1. Compounds 1 and 2 are interconvertible (can undergo conversion) during their preparation and under certain chemical conditions. Each of these isomers can self-assemble to produce a closed, hollow self-assembled multimer structure according to the present embodiments. Both isomers are fairly rigid with a donor/acceptor pair at each pentagonal edge.

More specifically, in the example of a lactam, it is comprised of an amine (a hydrogen bond donor) and a carbonyl (a hydrogen bond acceptor). When two identical lactam moieties interact, they may form a double hydrogen bond interaction between the amine on one lactam and the carbonyl on the other lactam, which constitute a binding pair, and the reversed interaction between the other binding pair.

Compound 3 is a γ-lactam and a structural isomer of Compound 1, wherein the position of the carbonyl and amine are switched and with potential interconversion into the δ-lactam Compound 4, depending on the chemical conditions. Compounds 5-7 possess a δ-lactam associating group, which is less rigid than the γ-lactams.

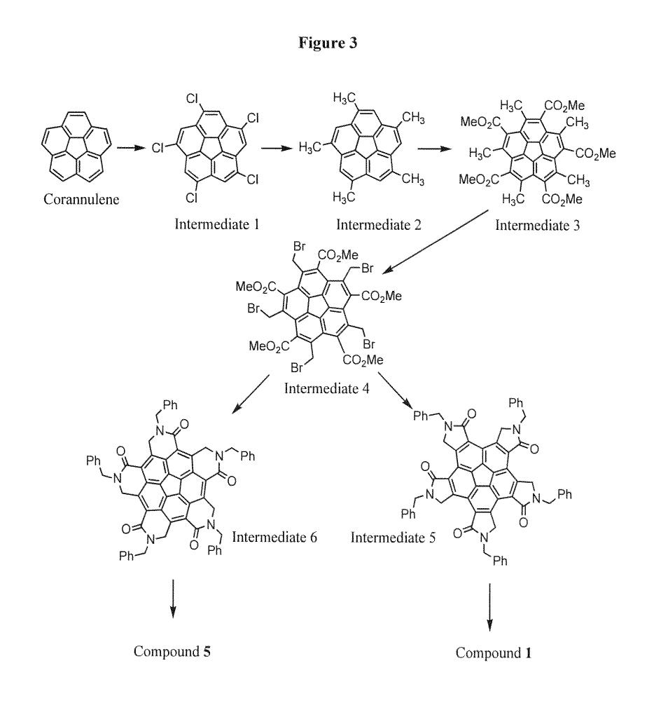

An exemplary synthetic pathway for preparing exemplary structurally symmetric compounds that can serve as chemical monomers for forming a self-assembled multimer structure is presented in FIG. 3.

FIG. 3 presents a basic outline of the rudimentary syntheses on the route to obtaining diverse potential structurally symmetric chemical monomers according to the present embodiments, such as Compounds 1 and 5 (see, FIG. 1). As can be seen in FIG. 3, chlorination of corannulene with iodine monochloride yields sym-pentachlorocorannulene (Intermediate 1) thus providing a starting point for further synthetic transformations. The systematic conversion of Intermediate 1 into a variety of sym-pentasubstituted corannulene derivatives allows for the appropriate placement of the desired associating groups around the edge of the 5-fold symmetric aromatic core. The unidirectional substitution pattern in this intermediate allows for the synthesis of many pentagonal molecules with unidirectional substitution. As can be seen in FIG. 3, Intermediates 2-6 are intermediate compounds which form on the route towards the structurally symmetric chemical monomers presented in FIG. 1, and specifically Intermediate 5 yields Compound 1 and Intermediate 6 yields Compound 5.

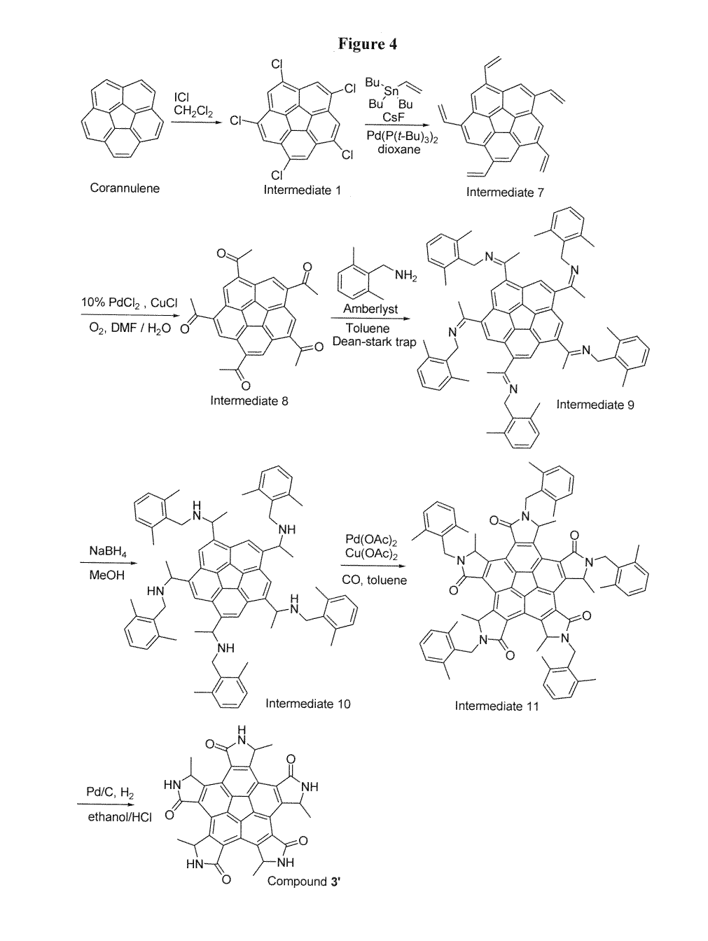

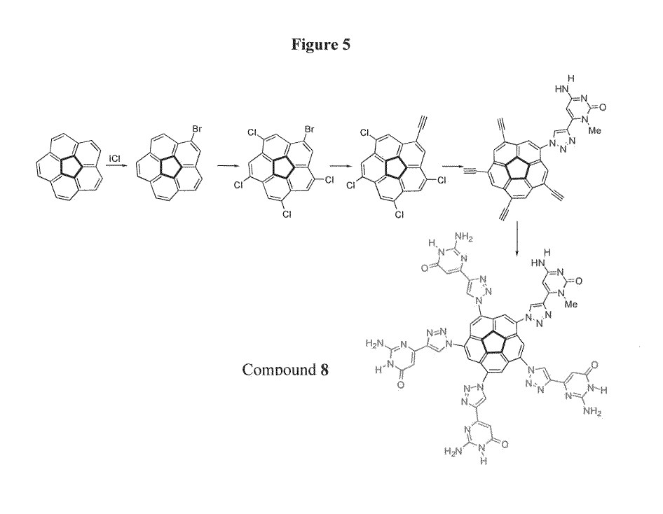

The preparation of Compound 3′, a derivative of Compound 3 having a methyl substituent on the γ-lactam associating group, is illustrated in FIG. 4. As can be seen in FIG. 4, 1,3,5,7,9-sym-pentachlorocorannulene (Intermediate 1) was carried out by reacting corannulene with iodine chloride ICl (1M in dichloromethane) according to the procedure described by Siegel and coworkers [Grube, G. H, Elliott, E. L, Steffens, R. J, Jones, C. S, Baldrige, K. K, Siegel, J. S, Org. Lett. 2003, 5, 713-716].

1,3,5,7,9-sym-pentavinylcorannulene (Intermediate 7) is afforded from Intermediate 1 by reaction with vinyltributylstannane in the presence of catalytic amounts of palladium bis(tri-tert-butylphosphine) and cesium fluoride in dioxane, according to the procedure described for other aromatic substrates by Fu and coworkers [Littke, A. F, Schwarz, L, Fu, G. C, J. Am. Chem. Soc. 2002 124, 6343-6348].

1,3,5,7,9-sym-pentaacylcorannulene (Intermediate 8) is afforded from Intermediate 7 by the Hoechst-Wacker oxidation process using catalytic amounts of palladium dichloride and cuprous chloride in DMF-water under oxygen atmosphere according to the method of Spencer and coworkers [Wright, J. A, Gaunt, M. J, Spencer, J. B, Chem. Eur. J. 2006, 12, 949-955].

1,3,5,7,9-sym-penta(2,6-dimethylbenzyliminoethyl)-corannulene (Intermediate 9) is afforded from Intermediate 8 by reaction with 2,6-dimethylbenzylamine while refluxing in toluene in the presence of amberlyst using azeotropic distillation with a Dean-Stark trap [Knettle, B. W, Flowers, R. A, Org. Lett. 2001, 3, 2321-2324].

1,3,5,7,9-sym-penta(2,6-dimethylbenzylaminoethyl)-corannulene (Intermediate 10) is afforded from Intermediate 9 by reductive amination with sodium borohydride in methanol.

1,3,5,7,9-sym-N-[2,6-dimethylbenzyl]pentalactam-corannulene (Intermediate 11) is afforded from Intermediate 10 by a palladium-catalyzed carbonylation reaction using palladium acetate and cupric acetate in toluene under CO atmosphere, according to the procedure of Tokuda and coworkers [Orito K, Horibata A, Nakamura T, Ushito H, Nagasaki H, Yuguchi S, Yamashita S, Tokuda M, J. Am. Chem. Soc. 2002 124, 6343-6348].

Finally, Compound 3′ is afforded from Intermediate 11 by reductive debenzylation of the nitrogen atoms under acidic conditions of palladium-catalyzed hydrogenolysis.

Tiles Having More Than One Type of Hydrogen Bonding Associating Groups:

While the compounds and synthetic pathways described hereinabove can be used to form a homo-multimer structure, composed of identical chemical monomers, hetero-multimer structures can also be prepared. Such compounds include two or more types of associating groups attached to the corannulene core.

FIG. 5 presents a chemical monomer, Compound 8, which can serve for forming such a hetero-multimer structure and a synthetic pathway for preparing same. As shown in FIG. 5, Compound 8 has four associating groups of one type and one compatible, yet different, associating group of another type. As can be seen in FIG. 5, the associating group drown in blue, albeit it offers a similar set of hydrogen bond donors and acceptors as the associating group drown in red, differs from the latter in the ratio of acceptors to donors in each set and in the way the various donors and acceptors are positions relatively to one another, hence these associating groups may fail to cross-associate and will self-assemble into a closed multimer provided three other types of structurally symmetric monomers will be present, as discussed below and presented in FIG. 2.

To demonstrate the possibility to construct a closed chemical hetero-multimer having more than one type of monomers, namely from two types of associating groups which are used to form four types of chemical monomers (tile), a virtual set of tiles was complied: one tile type, referred to herein as an “all-red” tile type, having 5 identical “red edges” each corresponding to one type of associating group, a second tile type, referred to herein as an “all-yellow” tile type, having 5 identical “yellow edges” each corresponding to the other type of associating group, a third tile type, referred to herein as an “4r1y” tile type, having four “red edges” and one “yellow edge”, and a fourth tile type, referred to herein as an “4y1r” tile type, having four “yellow edges” and one “red edge”.

As discussed hereinabove, FIG. 2 presents a schematic illustration of three combinatorial schemes for constructing a closed chemical hetero-multimer from four tile types having two edge types, wherein each edge is marked with red or yellow, and wherein the dashed lines connect two structurally complementary edges which are in contact in the resulting closed chemical hetero-multimer. As can be seen in FIGS. 2a-c, this system of four tile types can be arranged so as to form a closed chemical hetero-multimer in three patterns.

Preparation of Tiles Which Interact Via Metal Coordination:

The use of metal coordination chemistry offers a multitude of assembly as well as disassembly options. Attachment of chelating groups or polydentate groups at each of the corannulene edges creates a tile that cannot self-assemble by itself but can form very strong assemblies in the presence of the appropriate metal ions.

The chelating moiety is selected suitable for forming a stable complex with the desired metal. The stability of the metal-coordination complex typically depends on the number, type and spatial arrangement of the metal-coordinating groups surrounding the metal ion(s) and their fit to the coordination sphere of the metal.

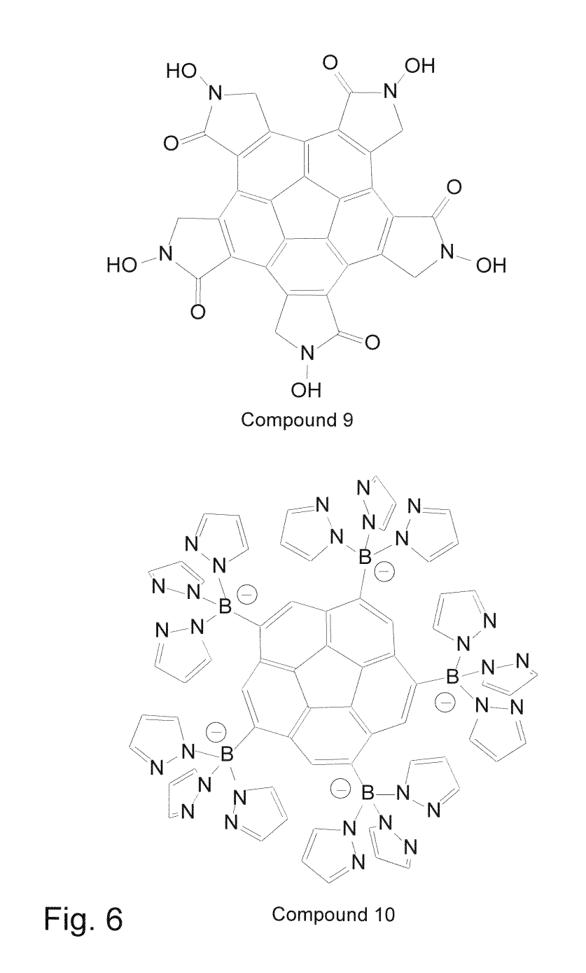

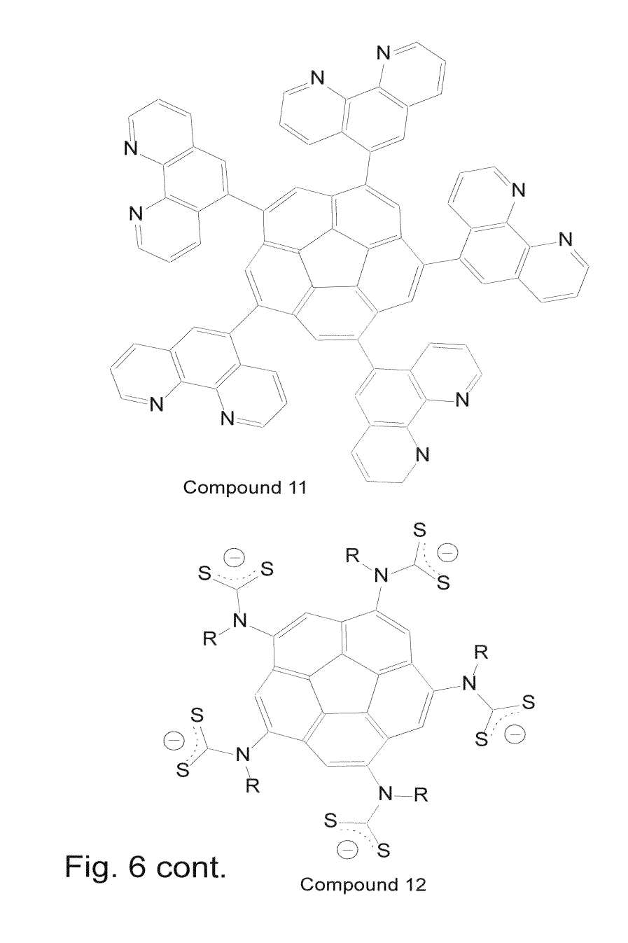

Non-limiting exemplary representatives of structurally symmetric compounds which can exploit the metal coordination binding properties are shown in FIG. 6 and include Compound 9 which exhibits hydroxamic acid associating groups around the edge of the corannulene, Compound 10 which exhibits trispyrazolylborate associative groups, Compound 11 which exhibits phenanthroline associating groups and Compound 12 which exhibits dithiocarbamate associating groups.

In each of these exemplary cases, the assembly of the tiles to a closed chemical multimer depends on the presence of a chemically suitable metal to form the metal coordinative bonds between two associating groups on two monomers. The type of metal should correspond to the type of chelator-type or polydentate associating groups.

Preparation of Tiles Which Interact Via Disulfide Bonds:

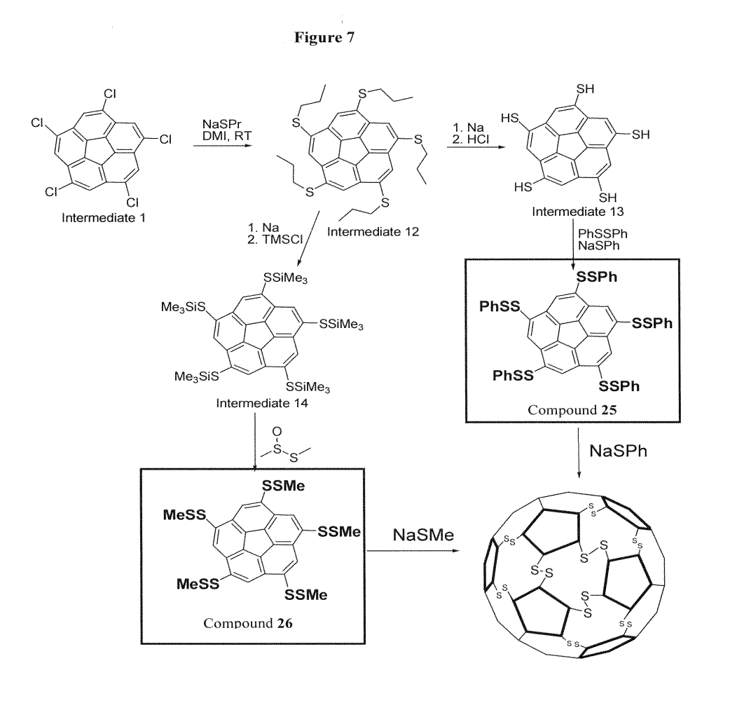

FIG. 7 presents a basic outline of the rudimentary syntheses on the route to obtaining diverse potential structurally symmetric chemical monomers which can associate via disulfide bridges, according to the present embodiments, such as Compounds 25 and 26. As can be seen in FIG. 7, the chlorinated corannulene 1,3,5,7,9-sym-pentachlorocorannulene (Intermediate 1) again provides the starting point for further synthetic transformations.

1,3,5,7,9-Pentakis(1-propylthio)corannulene (Intermediate 12) was afforded from Intermediate 1 according to the procedure described by Scott and coworkers [M. Bancu, A. K. Rai, P. Cheng, R. D. Gilardi, L. T. Scott, Synlett, 2004, 1, 173-176]. Intermediate 1 (100 mg, 0.236 mmol) and sodium 1-propanethiolate (347 mg, 3.55 mmol), prepared as above from 1-propanethiol and sodium in absolute ethanol, were stirred in 10 mL of 1,3-dimethylimidazolidin-2-one (DMEU) at room temperature under nitrogen for 2 days. During the reaction period the color of the mixture changed from light green to dark green. Thereafter the reaction mixture was added to 100 mL of toluene, and the resulting solution was washed twice with water and then with brine. The organic layer was dried with anhydrous MgSO₄ and concentrated under reduced pressure. The resulting crude product was separated by chromatography on silica gel using a mixture of hexane/CH₂Cl₂ (80/10) as eluent to give 56 mg (yield of 38%) of Intermediate 12 as a slightly oily brown material.

¹H NMR (400 MHz, CDCl₃): δ=7.89 (s, 5H), 3.12 (t, J=7.2 Hz, 10H), 1.79 (m, 10H), 1.09 (t, J=7.2 Hz, 15H).

¹³C NMR (100 MHz, CDCl₃): δ=136.71, 134.58, 131.29, 124.57, 37.18, 22.85, 13.91 ppm.

1,3,5,7,9-Pentakis-mercaptocorannulene (Intermediate 13) is afforded from Intermediate 12 by either nucleophilic attack with a sodium thiolate or by reductive cleavage with metallic sodium according to the procedure described in the art [L. Testaferri, M. Tiecco, M. Tingoli, D. Chianelli, M. Montanucci, Chem. Comm., 1983, 751-755; and F. Maiolo, L. Testaferri, M. Tiecco, M. Tingoli, J. Org. Chem., 1981, 46, 3070-3073].

1,3,5,7,9-Pentakis(phenyldisulfido)corannulene (Compound 25) is afforded from Intermediate 13 by reaction with diphenyl-disulfide and catalytic amount of sodium thiophenolate, according to known equilibrium under these conditions [G. M. Whitesides et al., J. Org. Chem., 1977, 42, 332-338; and G. M. Whitesides et al., J. Org. Chem., 1993, 58, 642-647].

1,3,5,7,9-Pentakis(trimethylsilylthio)corannulene (Intermediate 14) is afforded from Intermediate 12 by using metallic sodium followed by trimethylchlorosilane.

1,3,5,7,9-Pentakis(phenyldisulfido)corannulene (Compound 26) is afforded from Intermediate 14 by a reaction with dimethylthiosulfinate or dimethyl thiosulfonate, as described earlier by Menichetti and coworkers [G. Capozzi, A. Capperucci, A. Degl'Innocenti, R. Del Duce, S. Menichetti, Tet. Lett., 1989, 30, 2995-2998].

### Example 2

### Self-Assembled Chemical Multimer Structure

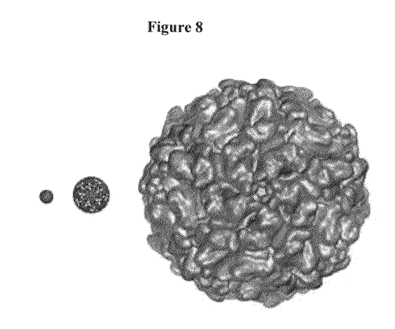

The structurally symmetric compounds presented and discussed above are design so as to have the capacity to self-assemble into hollow chemical dodecahedral spheroids (chemical multimers) according to the present invention. FIG. 8 presents a uni-scale size comparison between three different chemical spheroids, namely a fullerene (C₆₀) on the left, a self-assembled closed, hollow chemical multimer according to the present invention, comprised of 12 copies of Compound 1 in the middle, and a satellite tobacco mosaic virus (STMV), consisting of 60 identical copies of a single protein that make up the viral capsid (coating), on the right. As can be seen in FIG. 8, all three chemical structures constitute a spherical, closed chemical multimer and are known to be hollow, and span a rather large size range from 1.018 nm for C₆₀ outer diameter to 16 nm for STMV outer diameter.

In the case of the self-assembled closed, hollow multimer chemical structures according to the present invention, the size is directly derived from the size of the “tile”, or monomer, interchangeably referred to herein a structurally symmetric compound. Another factor that governs the final size of the present multimer chemical structures is the relative orientation of the tiles, which in turn is governed by the binding configuration between the associating groups.

The self-assembly of 12 pentagonal tiles to form a multimer chemical structure represents the arrival at the global energy minimum of the system. Achieving this minimum is not trivial because the system can generate many undesirable products that are less thermodynamically stable but are kinetically stable. For example, other aggregates that represent local minima but are insoluble in the medium could precipitate out of the solution and thereby exit the equilibrium, in which case the system does not reach the desired global minimum. Therefore, it is of crucial importance that the self-assembly of the tiles is maintained under conditions of uninterrupted equilibrium, allowing the system to reach the global minimum. In other words, the system should be thermodynamically stable and kinetically unstable. There are several approaches to achieve this condition, and the design of each approach should be based on the particular tiles and medium, and should comply with two basic conditions, namely (a) the formation of aggregates is highly reversible, and (b) all alternative aggregates are soluble.

In order to simplify the illustrations of the binding interactions between the tile-compounds in a closed and hollow chemical multimer dodecahedron, the illustrations include three tiles which represent a fourth of the entire dodecahedron.

Self-Assembly by Hydrogen-Bonds:

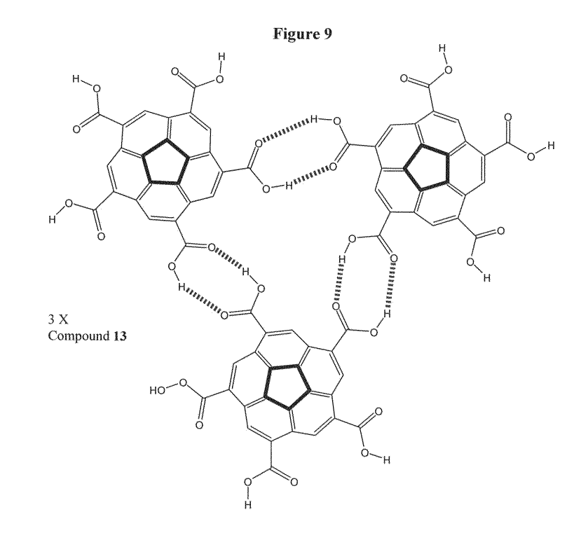

FIG. 9 presents an illustration of the relatively simple hydrogen-bond network connecting the tiles in a multimer made of an exemplary structurally symmetric monomer, namely Compounds 13, which interact with its identical counterparts. As can be seen in FIG. 9, each binding pair, consisting of one carboxylic group (an associating group) from two adjacent monomers, is characterized by two hydrogen bonds, giving rise to a stable self-assembled chemical multimer, yet this multimer may disassemble readily in conditions which do not favor the formation of hydrogen bonds, such as certain pH levels, solvents, ions and combination thereof.

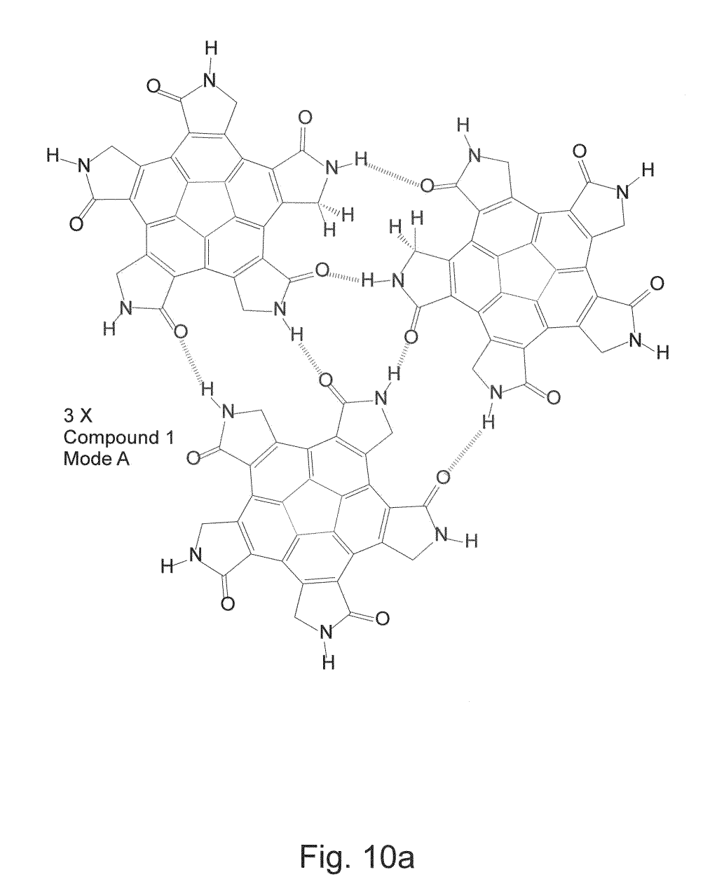

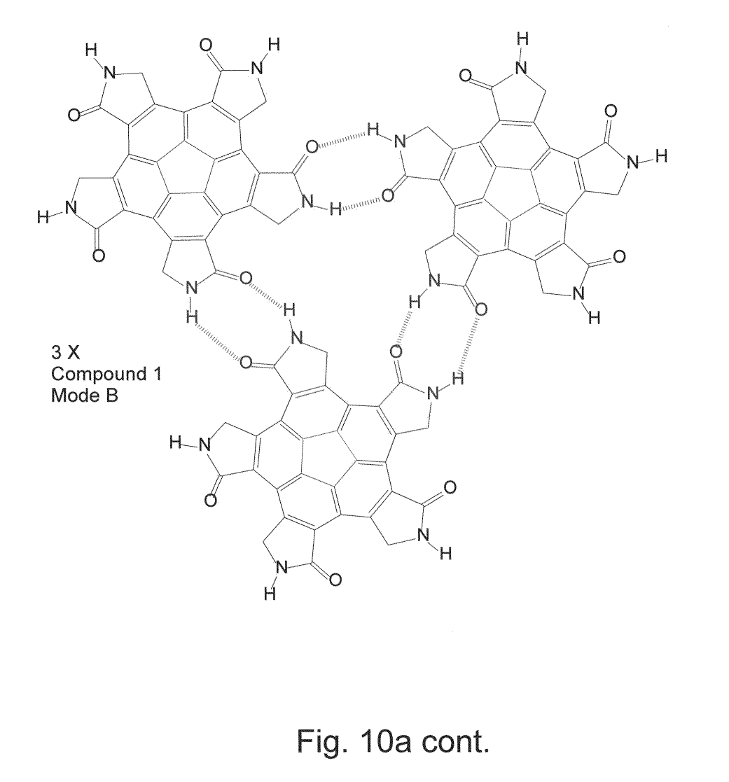

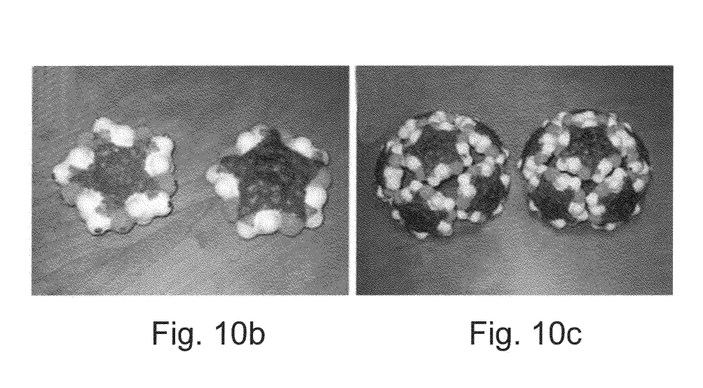

FIG. 10a presents a schematic illustration of two different modes of hydrogen bond network configurations which three sym-penta-γ-lactam-corannulene molecules (Compound 1) can form, referred to herein as Mode A and Mode B. As can be seen in FIG. 10a, these two possible hydrogen-bonding configurations give rise to two different “tiling” schemes wherein the tiles are skewed in one scheme with respect to the other, and are further distanced differently in the two schemes. As can be seen in FIG. 10a, one of these two binding configurations, or tiling schemes, is more compact, or tight than the other and therefore the binding configuration marked as “Mode A” will form a smaller spheroid than the spheroid afforded using the same compound but tiled by the binding configuration marked as “Mode B”.

FIG. 10b presents a photograph of two physical models of sym-penta-γ-lactam-corannulene (Compound 1, concave side up on the left and concave side down on the right) wherein magnets stand in place of hydrogen bonds. FIG. 10c presents a photograph of two physical models of dodecahedral hemispheres, each constructed from six identical magnet-fitted physical model units of Compound 1. As can be seen in FIG. 10c, the hemisphere on the right-hand side of the photograph, built according to the hydrogen bond network configuration Mode A is slightly smaller and more tightly packed than the hemisphere on the left-hand side of the photograph, built according to the hydrogen bond network configuration Mode B.

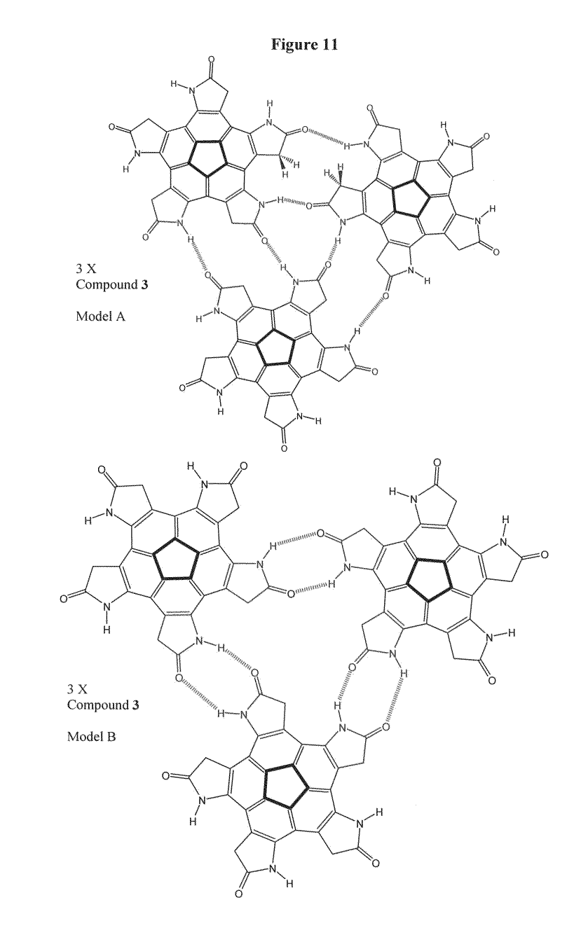

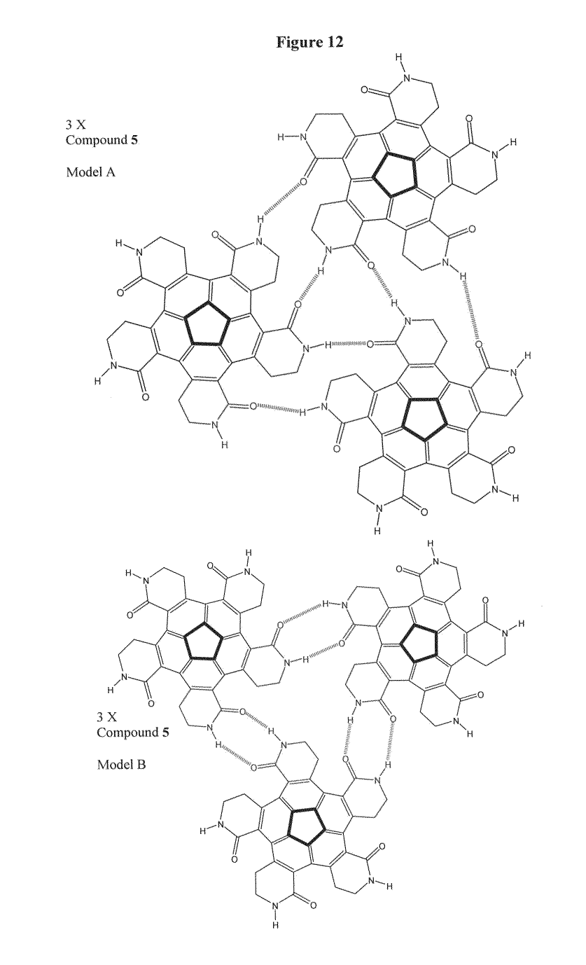

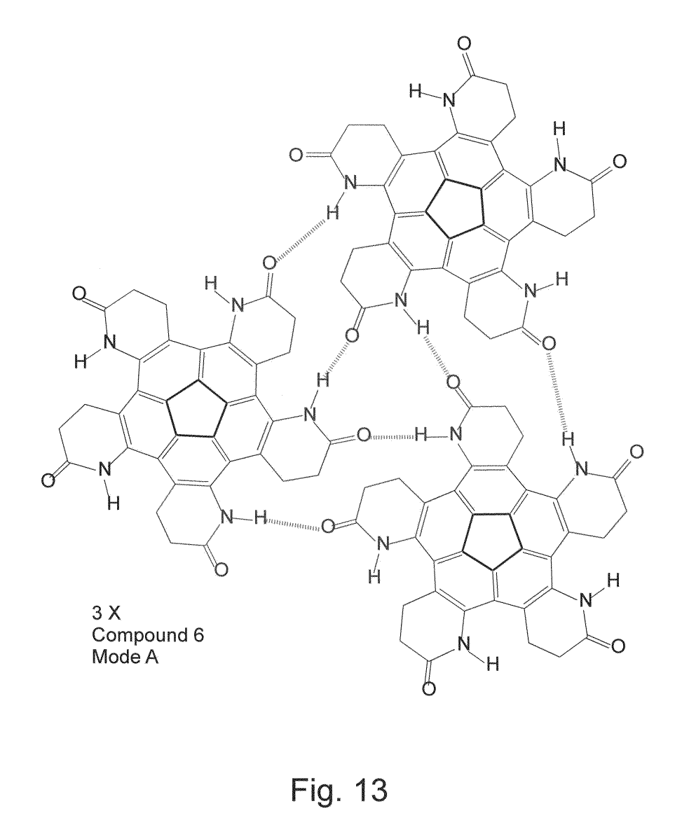

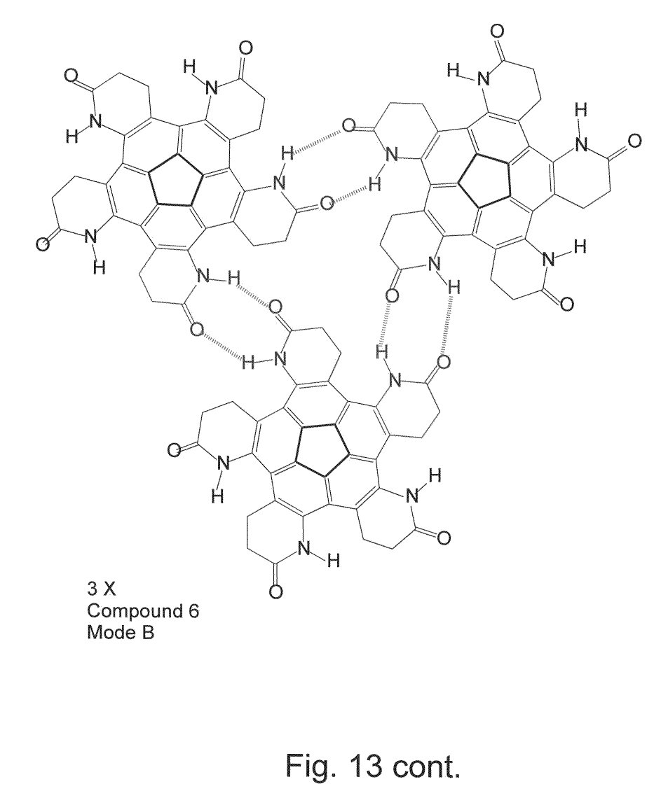

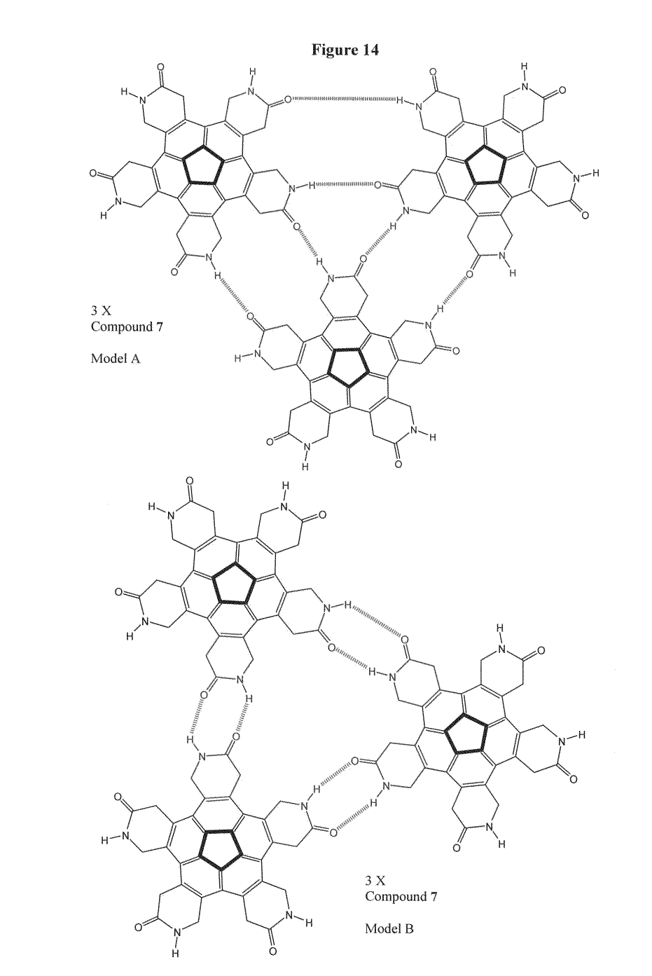

Similarly, FIGS. 11-14 present schematic illustrations of the two modes of hydrogen bond network configurations, referred to herein as Mode A and Mode B, which three structurally symmetric compounds, e.g., Compound 3 (FIG. 11), Compound 5 (FIG. 12), Compound 6 (FIG. 13) and Compound 7 (FIG. 14) can form.

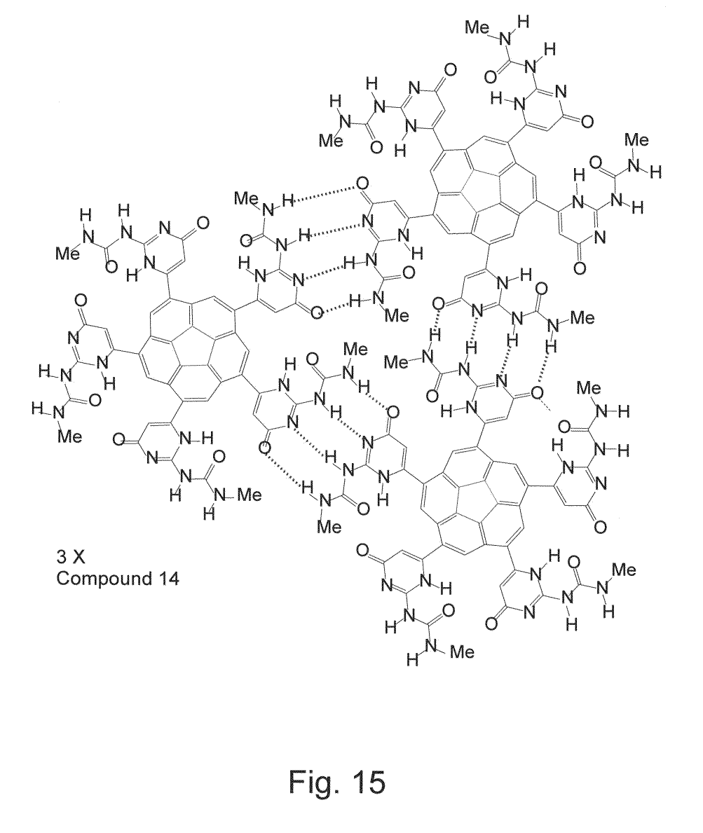

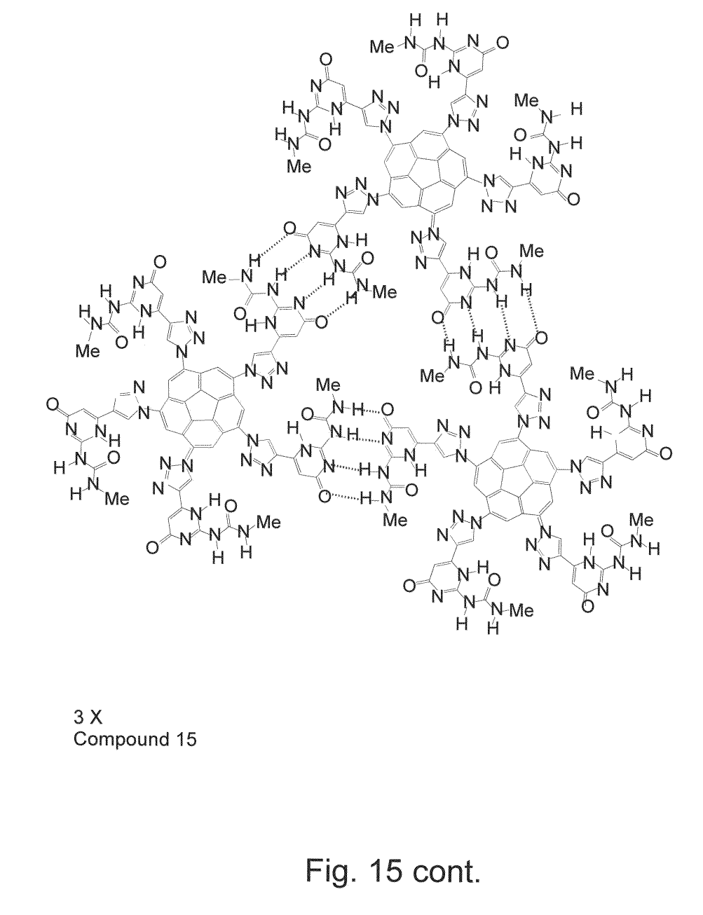

FIG. 15 presents an illustration of the intricate hydrogen-bond network connecting the tiles in a multimer made of two exemplary structurally symmetric monomers, namely Compounds 14-15, which interact each with its identical counterparts. As can be seen in FIG. 15, each binding pair, consisting of one associating group from two adjacent monomers, is characterized by four hydrogen bonds, giving rise to a very stable and robust self-assembled chemical multimer, yet this seemingly robust multimer may disassemble readily in conditions which do not favor the formation of hydrogen bonds, such as certain pH levels, solvents, ions and combination thereof.

Self-Assembly by Metal-Coordination:

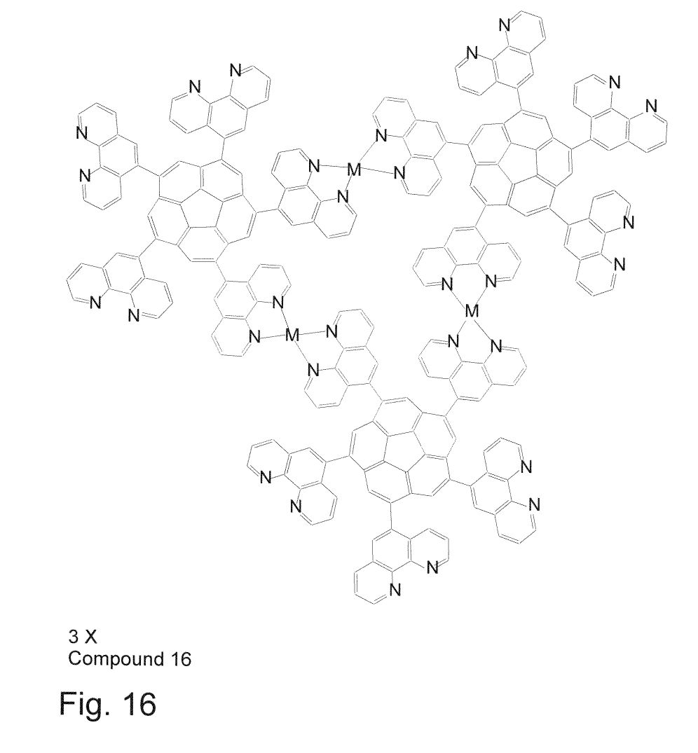

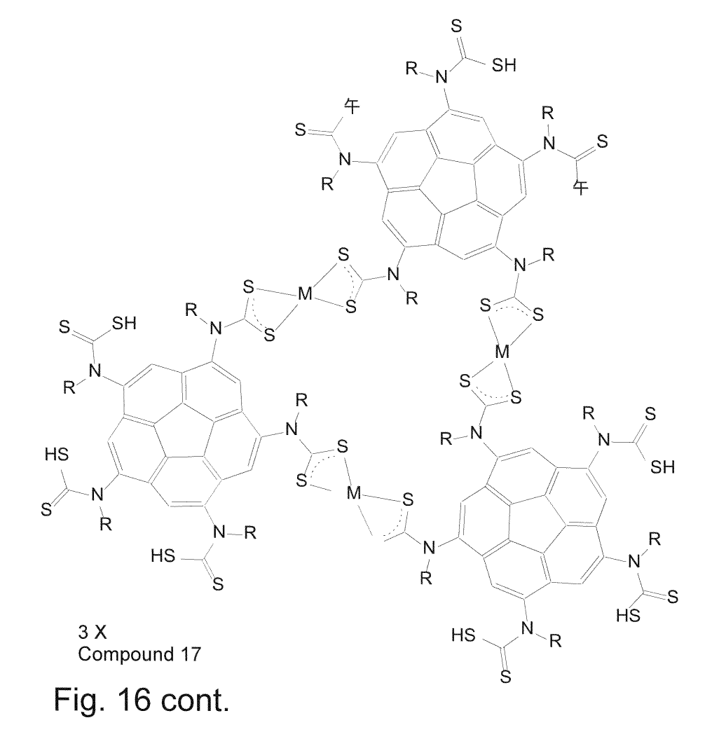

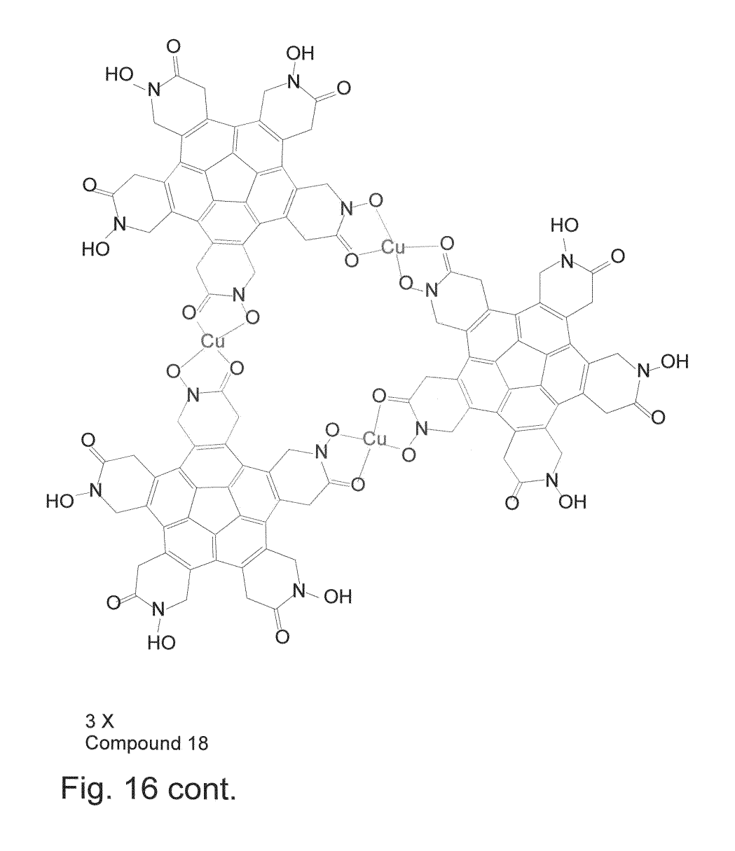

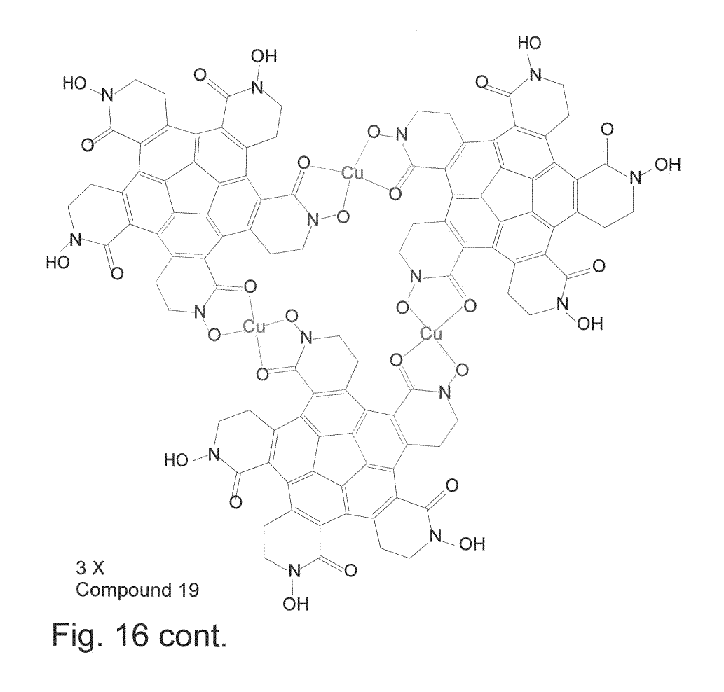

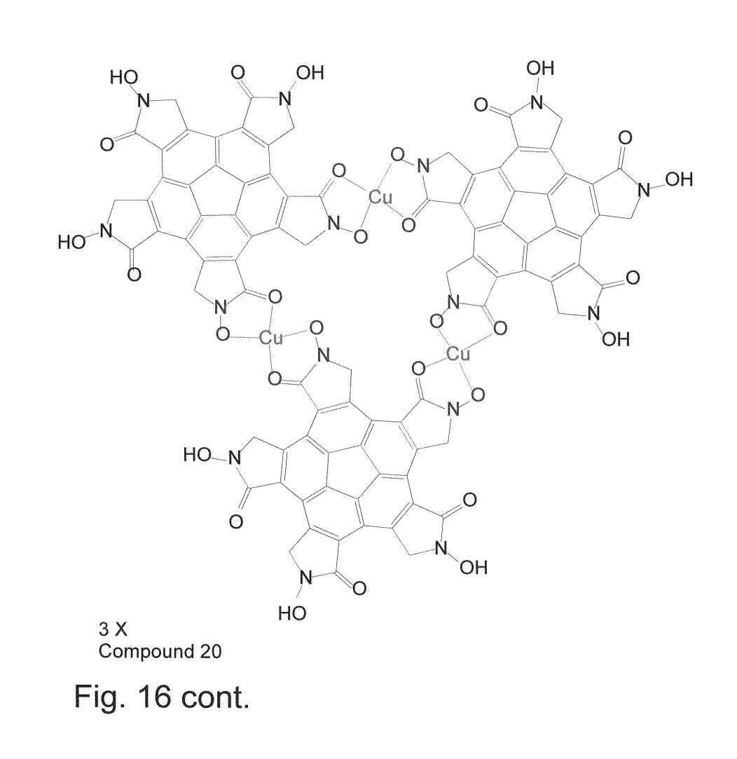

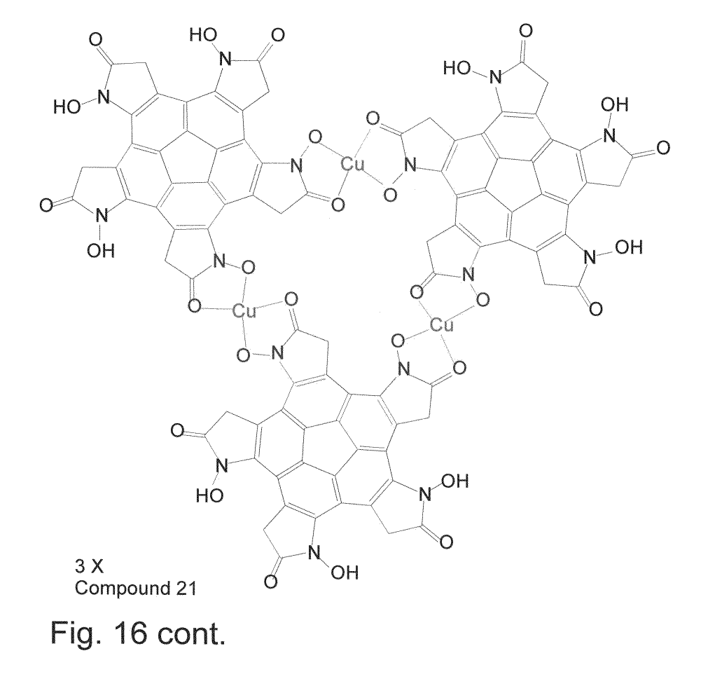

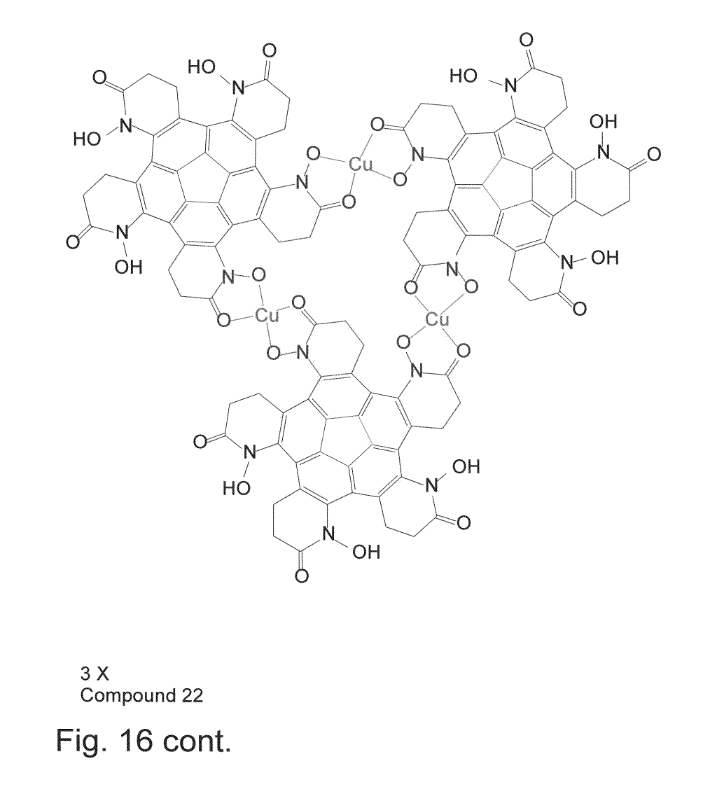

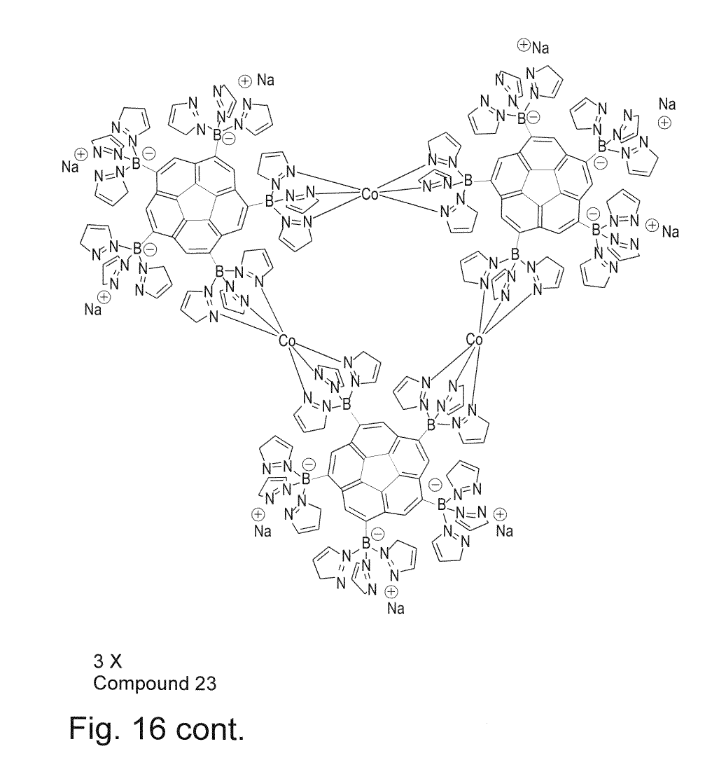

FIG. 16 presents an illustration of the assembly orientation of eight different exemplary chemical monomers, namely Compounds 16-23, which interact each with its identical counterparts via metal coordination binding. The appropriate choice of metals and ligands can offer very high binding constants, up to the equivalent of a covalent bond and yet these self-assembled chemical multimer structures would disintegrate back to the individual tiles under certain conditions such as the addition of chelators and other metal scavengers or otherwise depletion of the metal ions from the medium, certain pH levels and certain oxidation/reduction conditions which alter the oxidation state of the coordinating metal.

Self-Assembly by Electrostatic (Ionic) Bonds:

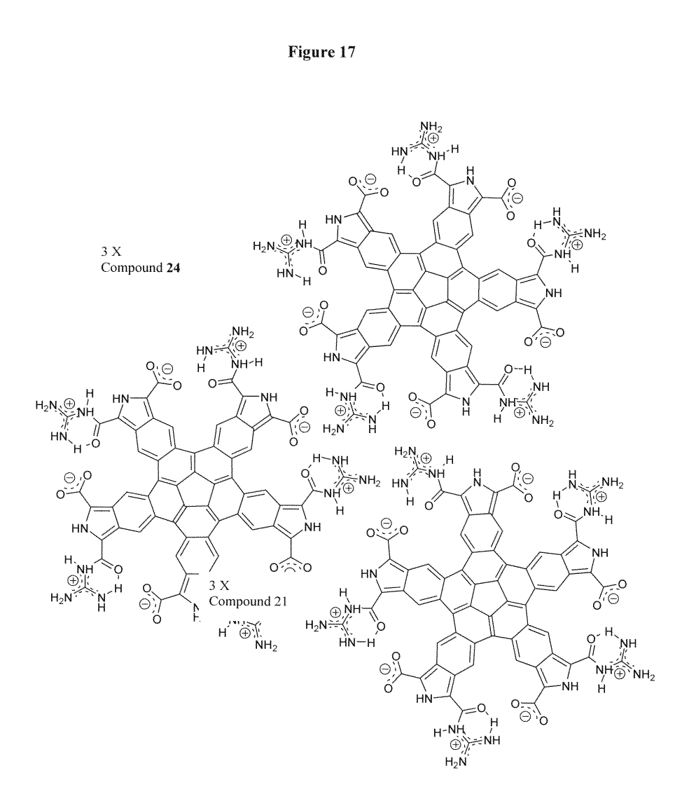

FIG. 17 presents an illustration of the assembly orientation of an exemplary chemical monomer, namely Compound 24, which interacts each with its identical counterparts via electrostatic or salt-bridge binding. Albeit strong, the electrostatic bond is highly sensitive to many chemical factors which can alter the bonding constant, such as ionization strength, pH, various solvent effects, etc. Many associating groups may be used to prepare chemical monomers for the assembly of chemical multimers which are based on salt-bridge binding. Among such associating groups are several naturally occurring ionizable moieties such as the side chains of the amino acids aspartate and glutamate.

Self-Assembly by Disulfide Bonds:

An example of a self-assembly process that is based on a thermodynamically stable and kinetically unstable system is based on the formation of disulfide bonds between two adjacent pentagonal tiles. Since dialkyl (or diaryl) disulfides undergo facile exchange with alkyl (or aryl) thiolates, a mixture of disulfides is expected to undergo rapid exchange in the presence of catalytic amounts of a thiolate anion, as illustrated in Scheme 1.

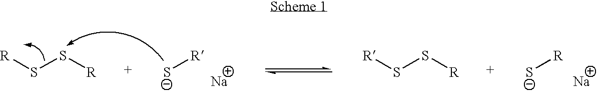

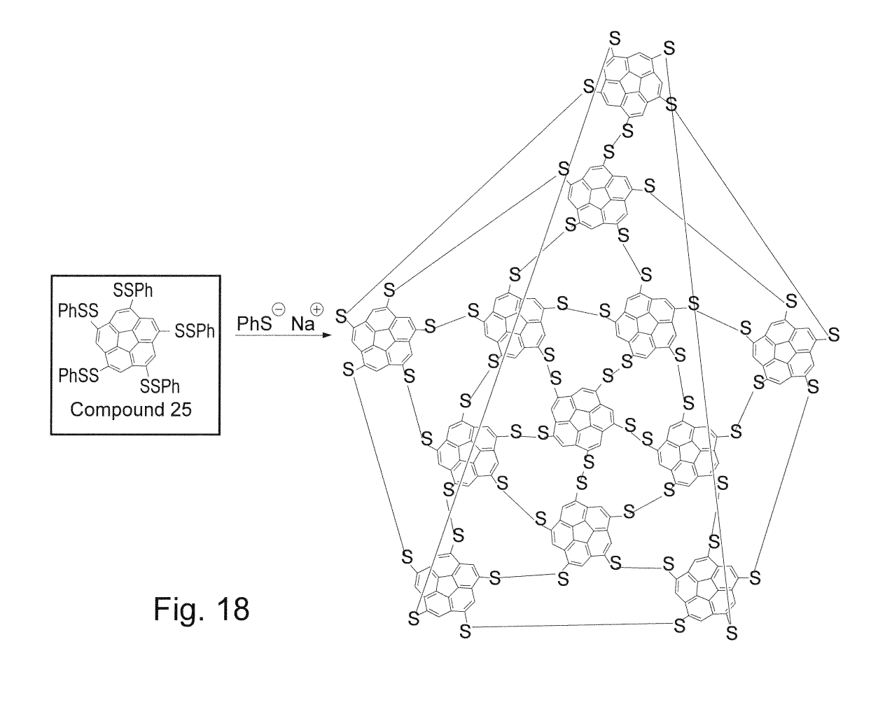

The reversible formation of disulfides between dialkyl or diaryl disulfides and alkyl- or aryl-thiolates serves as the basis for the formation of a multimer structure from structurally symmetric compound having thiol associating groups, as shown in FIGS. 7 and 18.

As can be seen in FIG. 7, an exemplary disulfide-based multimer structure can be obtained either directly from Intermediate 13 by reaction with diphenyl disulfide and a catalytic amount of sodium thiophenolate, according to known equilibrium under these conditions [G. M. Whitesides et al., J. Org. Chem., 1977, 42, 332-338; and G. M. Whitesides et al., J. Org. Chem., 1993, 58, 642-647], or by first isolating Compound 25, and then exposing Compound 25 to the equilibration conditions under catalysis of sodium thiophenolate.

An alternative pathway to the same disulfide-based multimer structure involves the use of Compound 26 by reaction thereof with sodium methylthiolate. The advantage of using this route, as compared with the route that is based on Compound 25, is that with Compound 26 the by-product, dimethyldisulfide is volatile under the reaction conditions and therefore the equilibrium is pushed towards the formation of a multimer structure.

FIG. 18 presents an illustration of the assembly process of an exemplary chemical monomer, namely Compound 25, which interacts with its identical counterparts via binding of two sulfur atoms, each from a different monomer, so as to form a disulfide bridge in the presence of an alkyl-thiolate salt or an aryl-thiolate salt, for example, sodium benzenethiolate. The reaction in the presence of the alkyl-thiolate or an aryl-thiolate salt forms the thermodynamically stable and kinetically unstable equilibrium. FIG. 18 further illustrates the disulfide bridging scheme between all twelve monomers which form one stable self-assembled dodecahedral chemical multimer (dodecamer). Albeit strong, disulfide bridges are highly sensitive to various specific chemical conditions and may form and break in the presence of, for example, particular redox reagents and radiation.

### Example 3

### Application of Self Assembly for Molecular Computation

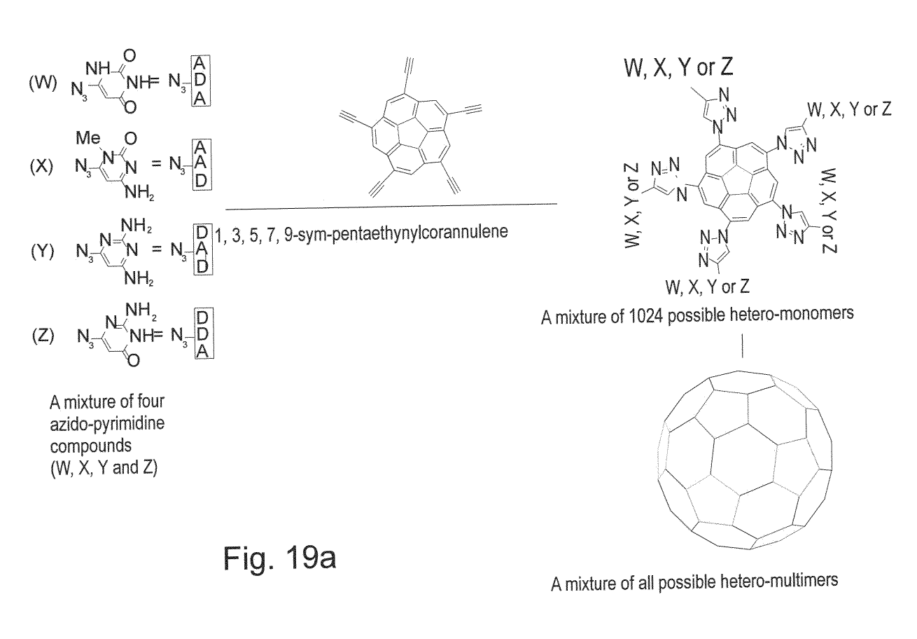

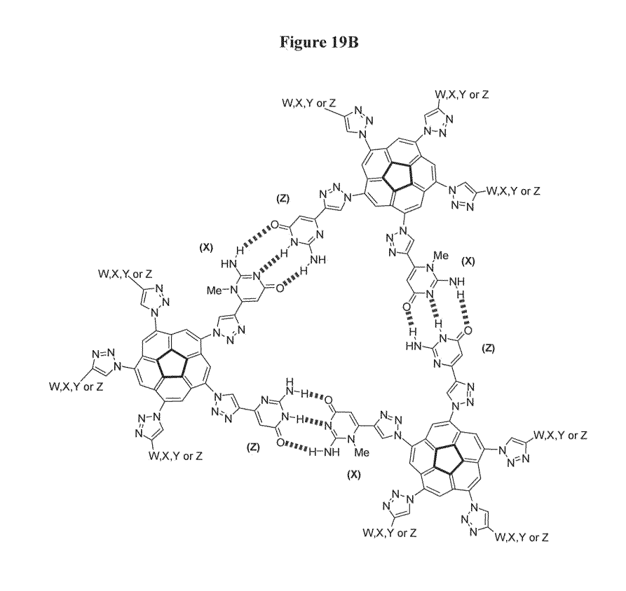

Self-assembled hetero-multimer structure systems can be used as molecular computing devices for solving mathematical problems. FIGS. 19a-b presents a schematic illustration of an exemplary self-assembled hetero-multimer structure system which can solve the mathematical problem of finding all the possible ways to cover a spherical surface using a library of 4⁵=1024 different pentagonal “tiles”, each having a random combination of 4 possible associating groups. As can be seen in FIG. 19a, this “mathematical problem” is solved chemically by synthesizing a library of 1024 different compounds in a one pot reaction of 1,3,5,7,9-sym-pentaethynylcorannulene with a uni-proportional mixture of four azido-pyrimidine derivative compounds, referred to herein by the letters W, X, Y and Z, wherein X and Z are compatible for binding by forming three parallel hydrogen bonds therebetween, and similarly so are W and Y. FIG. 19b presents a schematic illustration of the hydrogen bond network which forms in one example of a multimer structure which can assemble out of the combinatorial exercise, wherein the “letters” in the monomers are X and Z. A chemical analysis of the entire collection of all resultant multimer structures will provide the solution to the above-stated mathematical problem.

### Example 4

### Functionalization of Chemical Multimer Structures

The chemical multimer structure is further functionalized by means of an additional reactive moiety attached to the core and/or to any one of the associating groups, which can be utilized for its conjugation with various reactive moieties in other substances and active agents, as discussed in detail hereinbelow.

### Example 5

### Metal Coating of Chemical Multimer Structures

Functionalized chemical multimer structures are coated with one or more metals (metallized) by means of electroless deposition.

Chemical monomers can be functionalized so as to have one or more reactive moieties that can trigger the reduction of a metal ion into a metallic (elemental) state and further constitute a nucleation site for deposition of the same or other metallic elements. Self-assembly of such functionalized chemical monomers and subsequent homogeneous metal precipitation and coating by means of electroless deposition on the surface of the fully formed multimer structure (metallization) affords metal-coated chemical multimers.

Metal-coated chemical multimers, according to the present embodiments, can be regarded as nanoparticles which are characterized by uniform spherical shape and reproducible size, ranging from 2 nm to 6 nm.

A Silver-Coated Chemical Multimer Structure:

The chemical multimer structure is functionalized with reactive moieties which are a reducing moiety, such as aldehydes, hydrazines, hydrazides, and imines (Schiff-bases). Such reducing moieties have been found to act as mild reducing agents of silver ions, and therefore can affect an electroless deposition of silver on the surface of the chemical multimer presented herein.

FIG. 20 presents a schematic illustration of a silver-coating process effected on a chemical multimer structure according to the present embodiments, using an exemplary functionalized chemical monomer having an aldehyde (5-pentanalyl) reactive moiety attached to each of the γ-lactam associating groups (Compound 27). This reactive aldehyde moiety can be protected by means of an acetal or thioacetal during the synthesis of Compound 27 and also during the self-assembly thereof to a fully formed multimer structure. As can be seen in FIG. 20, once the multimer structure is formed, it is followed by deprotection of the aldehyde reactive moieties and the addition of soluble silver salt, such as silver nitrate, which leads to the formation of Intermediate 15 having nucleation of metallic silver on the surface of the multimer structure.

Such silver nuclei are known to induce further deposition of metallic silver under reducing condition such as, for example, the addition of glucose or sucrose to the aqueous solution, leading to the thickening of the metallic coating on the surface of the multimer structure and thus the formation of spherical metallic nanoparticles having a uniform size.

Aldehyde reactive moiety can serve as a reducing moiety for the process of silver-coating per-se, or be converted to another reducing moiety. As is well known and described in the art, aldehydes readily react with amines to form a Schiff base (imine), and the resulting imine can also serve as an effective reducing moiety according to the present embodiments.

More specifically, the process is effected as follows:

A functionalized chemical monomer having one or more reducing moieties such as aldehydes or imines attached thereto is provided, and a functionalized multimer structure self-assembles therefrom. The functionalized multimer structure self-assembles or dissolved in an aqueous solution.

An aqueous solution of silver ions is provided, and the functionalized multimer structure is brought in contact with the silver ions solution so as to effect reduction of the silver ions into elemental (metallic) silver atoms and thus deposition of metallic silver on the surface of the multimer structure is effected under reducing condition, such as in the presence of glucose or sucrose (sugar).

In the specific example of chemical multimer structure assembled from Compound 27 illustrated in FIG. 20, the outer diameter of the structure before coating it with metallic silver is about 2.5 nm, and after the silver-coating process the metallized chemical multimer structure can reach a uniform diameter of about 4 nm which is an optimal size for most known applications of metallic nanoparticles.

This process of silver deposition on the surface of the multimer structure is prolonged or repeated by replenishment of silver ions and reducing catalysts until the desired degree of metallization is achieved in terms of the thickness of the coat.

Palladium-, Nickel-, Cobalt- and/or Copper-Coated Chemical Multimer Structure:

Chemical multimer structures which are held together by metal-coordinating associating groups can be metallized by virtue of the presence of a metal in their composition. This metal can be reduced in-situ and serve as a catalytic metal for reduction of other metal ions, or as nuclei for deposition of additional metal atoms thereon as in the case of silver deposition presented hereinabove. Other chemical multimer structures can be functionalized with one or more chelating moieties which provide the means to attach catalytic metal ions to the surface of multimer structure.

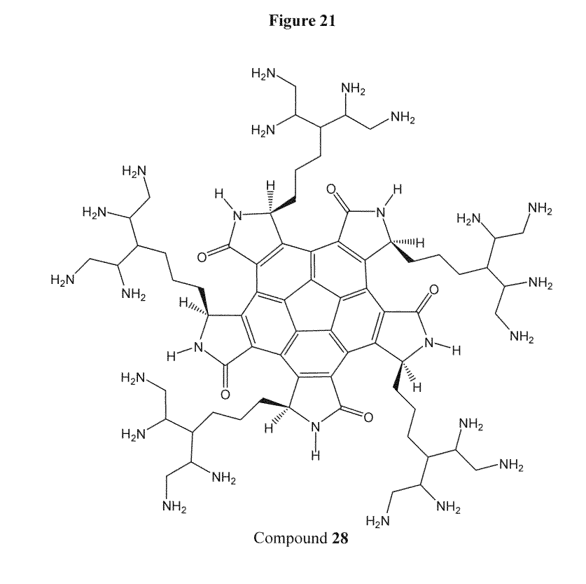

FIG. 21 presents an exemplary functionalized chemical monomer, Compound 28, having a tetradentate chelating moieties (3-propyl-pentane-1,2,4,5-tetraamine or two ethylenediamine groups on a propyl) attached to each of its associating groups. This tetradentate can serve as a chelating moiety for coordination of a catalytic metal ion.

This metal-coating of the chemical multimer is effected by:

(i) providing a multimer structure having at least one chelating moiety attached thereto, either as an associating groups or as additional reactive groups;

(ii) if the multimer structure does not contain catalytic metal ions as part of the binding of the monomers, the multimer structure is contacted with an aqueous solution containing ions of the catalytic metal to thereby obtain a complex of the functionalized multimer structure and the catalytic metal ions; and

(iii) contacting the complex of the functionalized multimer structure and the catalytic metal ions with a reducing agent which reduces the catalytic metal ions;

(iv) subsequent to or concomitant with the reduction process, the multimer structure, now having catalytic metal atoms on its surface, is contacted with another aqueous solution containing ions of the same metal or ions of a different metal, in the presence of a reducing agent, to thereby obtain the metal-coated multimer structure having one or more metals deposited on its surface.

### Example 6

### Chemical Multimer Structures Having an Active Agent Attached Thereto

As discussed hereinabove, the chemical multimer structures presented herein can be conjugated with one or more active agents by means of a reactive group which forms a part of the chemical monomers comprising the structure.

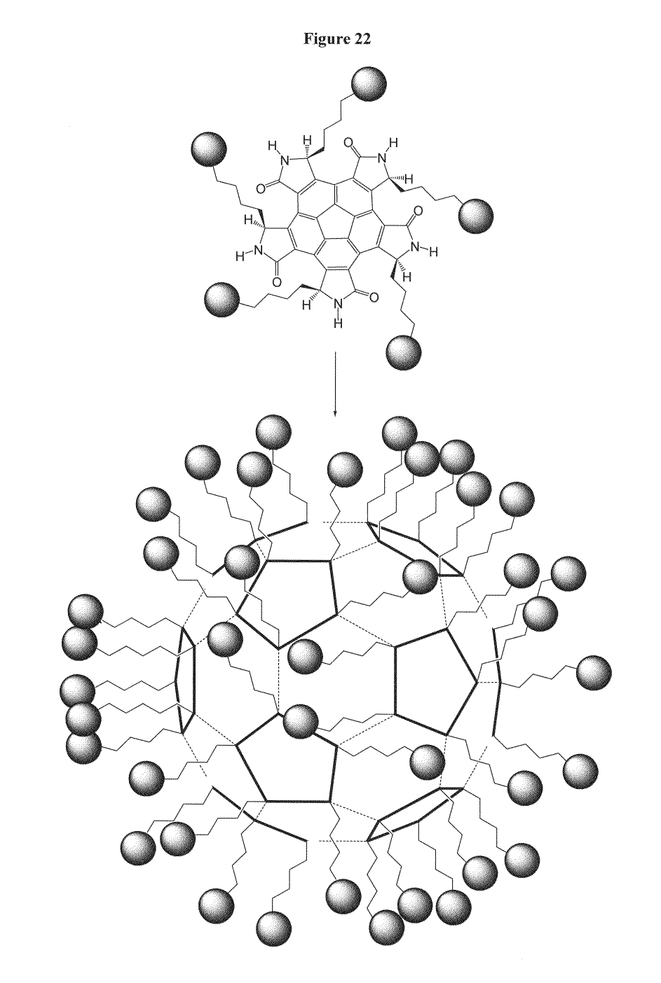

FIG. 22 presents a schematic illustration of a chemical monomer having an active agent (represented by a gray sphere) attached thereto, and the resulting multimer structure. According to this exemplary illustration, each monomer has 5 active agent moieties attached thereto and hence the resulting multimer structure has 12×5=60 active agent moieties attached thereto.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of creating a closed, hollow and self-assembled chemical multimer structure having a dodecahedral morphology, the method comprising:
(a) providing a plurality of chemical monomers that form the self-assembled chemical multimer structure, wherein each of said chemical monomers comprises a structurally symmetric core having a 5-fold rotational symmetry, and a plurality of at least one type of associating groups, said plurality of associating groups being symmetrically positioned at a periphery of said structurally symmetric core, whereas said chemical monomers have structural complementarity to one another so as to form the closed, hollow and self-assembled chemical multimer structure upon occurrence of an associative proximity and orientation of said associating groups; and
(b) subjecting said plurality of said chemical monomers to conditions allowing said chemical monomers to associate therebetween via said associating groups, thereby creating the closed, hollow and self-assembled chemical multimer structure.

2. The method of claim 1, wherein said chemical monomers in said plurality of chemical monomers are identical to one another.

3-5. (canceled)

6. The method of claim 1, wherein said plurality of chemical monomers comprises at least two different chemical monomers.

7. (canceled)

8. The method of claim 1, wherein said plurality of chemical monomers comprises from two to four types of chemical monomers that are different from one another.

9. (canceled)

10. The method of claim 1, wherein said associating groups are selected capable of forming a directional (polar) bond.

11. The method of claim 1, where said associating groups are selected capable of forming a chemically-reversible bond.

12-14. (canceled)

15. The method of claim 1, wherein said structurally symmetric core comprises at least one aromatic moiety.

16. The method of claim 15, wherein said structurally symmetric core comprises a corannulene moiety.

17. (canceled)

18. A closed, hollow and self-assembled chemical multimer structure having a dodecahedral morphology comprising a plurality of chemical monomers, wherein each of said chemical monomers comprises a structurally symmetric core having a 5-fold rotational symmetry, and a plurality of at least one type of associating groups, said plurality of associating groups being symmetrically positioned at a periphery of said structurally symmetric core, whereas said chemical monomers have structural complementarity to one another, thus forming the closed, hollow and self-assembled chemical multimer structure via associative proximity and orientation of said associating groups.

19. The chemical multimer structure of claim 18, wherein said chemical monomers in said plurality of chemical monomers are identical to one another.

20-22. (canceled)

23. The chemical multimer structure of claim 18, wherein said plurality of chemical monomers comprises at least two different chemical monomers.

24. (canceled)

25. The chemical multimer structure of claim 18, wherein said plurality of chemical monomers comprises from two to four types of chemical monomers that are different from one another.

26-27. (canceled)

28. The chemical multimer structure of claim 18, wherein said structurally symmetric core comprises at least one aromatic moiety.

29. The chemical multimer structure of claim 28, wherein said structurally symmetric core comprises a corannulene moiety.

30. A composition comprising the closed, hollow and self-assembled chemical multimer structure of claim 18 and at least one active agent being attached to and/or encapsulated in said chemical multimer structure.

31. (canceled)

32. The composition of claim 30, wherein said at least one active agent is selected from the group consisting of a pharmaceutically active agent, a labeling agent, a surface-modifying agent, a chemical compound, a metal and a nanoparticle.

33. The composition of claim 32, wherein said pharmaceutically active agent is selected from the group consisting of a therapeutically active agent and a targeting moiety.

34. The composition of claim 33, comprising at least one therapeutically active agent encapsulated in or attached to said multimer structure and at least one targeting moiety attached to said multimer structure.

35. The composition of claim 34, wherein said associating groups in said chemical monomers forming said multimer structure are selected capable of forming a biocleavable bond.

36-39. (canceled)

40. The composition of claim 33, comprising at least one therapeutically active agent attached to said multimer structure, said at least one therapeutically active agent being an epitope.

41. The composition of claim 40, comprising a plurality of said epitopes.

42. (canceled)

43. The composition of claim 32, wherein said active agent is a labeling agent, the composition being identified for use in diagnosis.

44-45. (canceled)

46. The composition of claim 32, wherein said active agent is a chemical compound, said chemical compound being encapsulated in said multimer structure.

47. The composition of claim 46, being identified for use in an analytical method for determining a chemical feature of said chemical compound.

48-49. (canceled)

50. The composition of claim 32, wherein said active agent is a nanoparticle, said nanoparticle being encapsulated in said multimer structure.

51. A method of delivering an active agent selected from the group consisting of a therapeutically active agent and a labeling agent to a bodily site of a subject in need thereof, the method comprising administering to the subject a composition which comprises the closed, hollow and self-assembled chemical multimer structure of claim 18 and said active agent, said active agent being attached to and/or encapsulated in said chemical multimer structure.

52. The method of claim 51, wherein said chemical multimer structure further comprises a targeting moiety attached thereto.

53-54. (canceled)

55. A method of immunization comprising administering to a subject a composition which comprises the closed, hollow and self-assembled chemical multimer structure of claim 18 and a plurality of epitopes, said plurality of epitopes being attached to said chemical multimer structure.

56. A plastic crystal comprising a plurality of the closed, hollow and self-assembled chemical multimer structure of claim 18.

57-58. (canceled)

59. A method of preparing a composition which comprises the closed, hollow and self-assembled chemical multimer structure of claim 18 and an active agent being encapsulated in the chemical multimer structure, the method comprising:
(a) providing said plurality of chemical monomers that form the self-assembled chemical multimer structure, wherein each of said chemical monomers comprises a structurally symmetric core and a plurality of at least one type of associating groups, said plurality of associating groups being symmetrically positioned at a periphery of said structurally symmetric core, whereas said chemical monomers have structural complementarity to one another so as to form the closed, hollow and self-assembled chemical multimer structure upon occurrence of an associative proximity and orientation of said associating groups; and
(b) subjecting said plurality of said chemical monomers to conditions allowing said chemical monomers to associate therebetween via said associating groups in the presence of said active agent, thereby creating the closed, hollow and self-assembled chemical multimer structure having said active agent encapsulated therein.

60. A method of preparing a composition which comprises the closed, hollow and self-assembled chemical multimer structure of claim 18 and an active agent attached to the chemical multimer structure, the method comprising:
(a) providing said plurality of chemical monomers that form the self-assembled chemical multimer structure, wherein each of said chemical monomers comprises a structurally symmetric core and a plurality of at least one type of associating groups, said plurality of associating groups being symmetrically positioned at a periphery of said structurally symmetric core, whereas said chemical monomers have structural complementarity to one another so as to form the closed, hollow and self-assembled chemical multimer structure upon occurrence of an associative proximity and orientation of said associating groups;
(b) attaching said active agent to at least one of said chemical monomers, to thereby obtain a plurality of said chemical monomers in which at least of said chemical monomers has said active agent attached thereto; and
(c) subjecting said plurality of said chemical monomers in which at least of said chemical monomers has said active agent attached thereto to conditions allowing said chemical monomers to associate therebetween via said associating groups in the presence of said active agent, thereby creating the closed, hollow and self-assembled chemical multimer structure having said active agent attached thereto.

61. A method of preparing a composition which comprises the closed, hollow and self-assembled chemical multimer structure of claim 18 and an active agent attached to the chemical multimer structure, the method comprising:
(a) providing said plurality of chemical monomers that form the self-assembled chemical multimer structure, wherein each of said chemical monomers comprises a structurally symmetric core and a plurality of at least one type of associating groups, said plurality of associating groups being symmetrically positioned at a periphery of said structurally symmetric core, whereas said chemical monomers have structural complementarity to one another so as to form the closed, hollow and self-assembled chemical multimer structure upon occurrence of an associative proximity and orientation of said associating groups;
(b) subjecting said plurality of said chemical monomers in which at least of said chemical monomers has said active agent attached thereto to conditions allowing said chemical monomers to associate therebetween via said associating groups in the presence of said active agent, thereby creating the closed, hollow and self-assembled chemical multimer structure; and
(b) attaching said active agent to closed, hollow and self-assembled chemical multimer structure, thereby creating the closed, hollow and self-assembled chemical multimer structure having said active agent attached thereto.

62. A method of preparing a chemical monomer capable of forming a closed, hollow and self-assembled multimer structure having a dodecahedral morphology, the method comprising:
providing a structurally symmetric core compound having a 5-fold rotational symmetry;
symmetrically attaching at a periphery of said core compound a plurality of at least one type of associating groups,
wherein said chemical monomer and said associating groups are selected such that said chemical monomer has a structural complementarity to an identical and/or different chemical monomer, which allows forming the closed, hollow and self-assembled multimer structure upon occurrence of associative proximity and orientation of said associating groups.

63. (canceled)

64. The method of claim 62, wherein said structurally symmetric core comprises at least one aromatic moiety.

65. The method of claim 64, wherein said structurally symmetric core comprises a corannulene moiety.

66. A compound having a structurally symmetric core having a 5-fold rotational symmetry, and a plurality of at least one type of associating groups symmetrically positioned at a periphery of said core.

67. (canceled)

68. The compound of claim 66, wherein said structurally symmetric core comprises at least one aromatic moiety.

69. The compound of claim 68, wherein said structurally symmetric core comprises a corannulene moiety.

70. A metal-coated chemical multimer structure comprising the closed, hollow and self-assembled chemical multimer structure of claim 18 and at least one metal deposited over at least a portion of the surface thereof.
